(19) United States
(12) Patent Application Publication
(10) Pub. No.: US 2025/0026707 A1
(43) Pub. Date: Jan. 23, 2025

(54) METHOD FOR CONVERTING ONE OR MORE HYDROCARBONS, AND A CATALYST USED THEREFOR

(71) Applicant: NEWCHEM21, INC., San Francisco, CA (US)

(72) Inventors: Sagar GADEWAR, San Francisco, CA (US); Zhenhua ZHOU, San Francisco, CA (US); Madhura KELKAR, San Francisco, CA (US); Vivek JULKA, San Francisco, CA (US); Carlitta DAVIS, San Francisco, CA (US)

(21) Appl. No.: 18/714,016

(22) PCT Filed: Nov. 29, 2022
(86) PCT No.: PCT/US2022/080598

§ 371 Date: May 28, 2024

Related U.S. Application Data
(60) Provisional application No. 63/283,819, filed on Nov. 29, 2021.

(51) Int. Cl. C07C 51/215; B01D 3/14; B01D 3/36; B01J 21/06; B01J 23/652; B01J 37/08; C07C 51/46

(52) U.S. Cl. CPC C07C 51/215; B01D 3/143; B01D 3/36; B01J 21/063; B01J 23/6525; B01J 37/08; C07C 51/46

(57) ABSTRACT

A method for converting one or more hydrocarbons includes feeding a fluid comprising one or more light alkanes to a reactor system, and producing one or more oxygenates from the one or more light alkanes in the reactor system. The reactor system comprises a reactor containing at least one catalyst, the at least one catalyst comprises one or more oxides of molybdenum, vanadium, niobium, cerium, titanium, zirconium, and one or more precious metals. An oxygenate productivity is higher than about 50 g/kg cat.h after 100 hours of time on stream.

### CROSS-REFERENCE TO RELATED APPLICATIONS

This application claims priority to and the benefit of U.S. Provisional Patent Application No. 63/283,819 filed on Nov. 29, 2021, and entitled, “METHOD FOR CONVERTING ONE OR MORE HYDROCARBONS, AND A CATALYST USED THEREFOR,” which is incorporated herein by reference in its entirety.

### STATEMENT REGARDING GOVERNMENTALLY SPONSORED RESEARCH OR DEVELOPMENT

None.

### BACKGROUND

The oxidative dehydrogenation (ODH) of light alkanes (C₁ to C₄) to make oxygenates is challenging, but potentially rewarding, and it could lead to a paradigm shift in the supply chain of several bulk chemicals. Unfortunately, despite the significant desire to selectively oxidize light alkanes under mild conditions, progress has been hampered due to its chemical inertness, which results from a high C—H bond strength, particularly for methane and ethane as shown in Table 1. Another crucial limitation arises from the fact that the partial oxidation products of ethane are inherently more reactive, with deep oxidation to COₓ (carbon monoxide (CO) and carbon dioxide (CO₂)) a limiting factor in the viability of catalytic systems. (Robert D. Armstrong, Graham J. Hutchings and Stuart H. Taylor, Catalysts 2016, 6, 71; Blanksby, S. J.).

TABLE 1
Dissociation energy of C—H bond and
charge on H of some light hydrocarbons

| | | C—H Bond E_{disoc} | |
|---|---|---|---|
| | Weakest | kilojoule per | Atomic |
| Hydrocarbon | C—H Bond | mole (kJ/mol) | Charge on H |
| Methane | Primary | 440 | +0.087 |
| Ethane | Primary | 420 | +0.002 |
| Propane | Secondary | 401 | −0.051 |
| Isobutane | Tertiary | 390 | −0.088 |

### SUMMARY

In some embodiments, a method for producing oxygenates from one or more light alkanes, the method comprises: providing a reactor system and a product separation system. The reactor system comprises at least one catalyst, the at least one catalyst comprises one or more oxides of molybdenum, vanadium, niobium, cerium, titanium, zirconium, and one or more precious metals. In some embodiments, a catalyst, comprises: an oxide of (Mo_{0.6}Nb_{0.22}V_{0.18})₅O₁₄.

In some embodiments, a method for converting one or more hydrocarbons, the method comprises: feeding one or more light alkanes to a reactor system. The reactor system comprises a reactor containing a catalyst, comprising the oxide of (MO_{0.6}Nb_{0.22}V_{0.18})₅O₁₄ comprised with a support.

In some embodiments, a method for converting one or more hydrocarbons, the method comprises: feeding one or more light alkanes to a reactor system. The reactor system comprises a reactor containing at least one catalyst. The at least one catalyst comprises one or more oxides of molybdenum, vanadium, niobium, cerium, titanium, zirconium, and one or more precious metals.

### BRIEF DESCRIPTION OF THE DRAWINGS

For a detailed description of the preferred embodiments of the invention, reference will now be made to the accompanying drawings in which:

FIG. 1 is a graphical depiction of an X-ray diffraction of a catalyst MoVNbOₓ/TiO₂ without containing a precious metal.

FIG. 2 illustrates a process flow diagram showing an overall process according to some embodiments.

FIG. 3 illustrates a schematic reactor layout according to some embodiments.

FIG. 4 illustrates another overall process flow diagram including a schematic reactor system and a liquid separation system according to some embodiments.

FIG. 5 illustrates still another overall process flow diagram according to some embodiments.

FIG. 6 illustrates yet another overall process flow diagram according to some embodiments.

FIG. 7 illustrates still yet another overall process flow diagram according to some embodiments.

FIG. 8 illustrates another overall process flow diagram according to some embodiments.

FIG. 9 illustrates a process flow for a separation process for the products of the reaction according to some embodiments.

FIG. 10 illustrates an overall process flow diagram for a reaction and separation scheme according to some embodiments.

FIG. 11 illustrates a schematic process flow diagram for a reaction and separation scheme according to some embodiments.

FIG. 12 illustrates another schematic process flow diagram for a reaction and separation scheme according to some embodiments.

FIG. 13 illustrates still another schematic process flow diagram for a reaction and separation scheme according to some embodiments.

FIGS. 14A and 14B illustrate schematic process flow diagrams for separation columns for the liquid product stream from the reactor in some embodiments.

FIG. 15 illustrates another schematic process flow diagram for a reaction and separation scheme according to some embodiments.

FIG. 16 illustrates still another schematic process flow diagram for a reaction and separation scheme according to some embodiments.

FIG. 17 illustrates yet another schematic process flow diagram for a reaction and separation scheme according to some embodiments.

FIG. 18 illustrates a schematic process flow diagram for a reaction and separation scheme according to some embodiments.

FIG. 19 is a chart showing the gas chromatograph (GC) results of Examples 7-15.

FIG. 20 illustrates a process flow diagram for a simulation of the overall process according to some embodiments.

FIG. 21 is a chart showing the stream properties for the streams illustrated in FIG. 20.

FIG. 22 illustrates a process flow diagram for a simulation of the overall process according to some embodiments.

FIG. 23 is a chart showing the stream properties for the streams illustrated in FIG. 22.

### DESCRIPTION

This present disclosure is directed to the method for converting one or more hydrocarbons using catalyst including one or more oxides of molybdenum, vanadium, and niobium, particularly an oxide of the formula (MO_{0.6}Nb_{0.22}V_{0.18})₅O₁₄ one or more precious metals, and one or more suitable supports along with separation techniques to enable continuous production of oxidation products. This disclosure includes reactor alternatives, catalyst alternatives, and reactants and products separation process alternatives.

As used herein, the term “precious metal” can include ruthenium, rhodium, palladium, osmium, iridium, platinum, or gold.

As used herein, the term “oxygenate” can mean a compound that includes oxygen as part of their chemical structure.

As used herein, the term “water” can mean water in a gas phase, and the water can include at least about 50%, at least about 99%, and at least about 99.9%, by weight, water based on the weight of water plus any impurities.

As used herein, the term “gas hourly space velocity” may be the ratio of the volumetric flow rate of gas to volume of catalyst bed.

As used herein, the terms “continuous stirred-tank reactor” may be abbreviated “CSTR”, “continuous catalyst regeneration” may be abbreviated “CCR”, “deionized” may be abbreviated “DI”, “degrees Celsius” may be abbreviated “C”, “percent” may be abbreviated “%”, “weight” may be abbreviated “wt”, “pounds per square inch” may be abbreviated “psi”, “gas hourly space velocity” may be abbreviated “GHSV”, “per hour” may be abbreviated “h-1”, and “gram produced per hour per kilogram of catalyst loaded” may be abbreviated “g/kg cat.h”.

As used herein, the term “upper portion” can refer to a top half or a top third of a column.

As used herein, the term “lower portion” can refer to the bottom half or the bottom third of a column.

As used herein, the term “central portion” or “middle portion” can refer to the central third of a column.

A classical system for alkane ODH is supported vanadium oxide with or without molybdenum. However, the reaction temperature with these catalysts is typically greater than 500° C. and produce mostly olefin instead of oxygenates (Miguel A. Bañares, Catalysis Today 51 (1999) 319-348). The use of catalysts based on oxides of molybdenum and vanadium together with other oxides of transition metals, e.g., Ti, Cr, Mn, Fe, Co, Ni, Nb, Ta or Ce, calcined at 400° C., was proposed by Thorsteinson, et al. in “The Oxidative Dehydrogenation of Ethane over Catalyst Containing Mixed Oxides of Molybdenum and Vanadium” (Journal of Catalysis, 52 (1978) 116). The catalysts are active at temperatures as low as 200° C. for the oxydehydrogenation of ethane to ethylene. One result was obtained over a solid with the composition M00.61V_{0.31}Nb_{0.08} supported in a gamma alumina, yielding a 30% of ethylene at 400° C. The optimum composition improves the activity of the metal oxides for ethane activation, but only small amount of acetic acid was made as by-product. Several U.S. Patents (U.S. Pat. Nos. 4,250,346; 4,524,236; 4,568,790; 4,596,787; and 4,899,003) have been granted on low temperature oxydehydrogenation of ethane to ethylene. U.S. Pat. No. 4,250,346 discloses the use of catalysts of the formula MoₕVᵢNbⱼAₖ in which A is Ce, K, P, Ni, and/or U, h is 16, i is 1 to 8, j is 0.2 to 10, and k is 0.1 to 5. U.S. Pat. No. 4,524,236 is directed to the use of a calcined catalyst of the formula MoₐV_{b}Nb_{c}Sb_{d}Xₑ. The above cited patents refer to other patents concerned with the production of ethylene from ethane by the oxydehydrogenation process and all refer to the formation of acetic acid as a by-product.

The gap between dioxygen activation, that is formation of reactive intermediates, and actual catalytic transformations, particularly of light hydrocarbons and alkanes under convenient aerobic conditions, using intrinsically stable inorganic catalysts, has not been effectively bridged. For example, using conventional molybdenum, vanadium, and oxygen (MoVO) system for aerobic oxidation of ethane to ethylene and acetic acid tends to form undesired ethylene, carbon monoxide, and carbon dioxide.

Further, a reported catalyst containing MoVNb promoted with phosphorus can produce a relatively higher yield of acetic acid as compared to unpromoted catalyst with the production of byproducts such as carbon monoxide, carbon dioxide and ethylene (U.S. Pat. No. 6,013,597). U.S. Pat. No. 6,030,920 reported an oxide catalyst comprising the elements Mo, V, Nb, and Pd. The novel catalytic system provides both higher selectivity and yield of acetic acid in the low temperature one step vapor phase direct oxidation of ethane with molecular oxygen containing gas without production of side products such as ethylene and CO. The role of palladium in the catalyst is like palladium catalyst in Wacker process which makes ethylene to acetaldehyde. The palladium-containing catalyst increase the acetic acid selectivity significantly. However, this type of catalyst typically has relatively low surface area which leads to low acetic acid productivity. Xuebing Li, and Enrique Iglesia reported a TiO₂ supported Mo_{0.61}V_{0.31}Nb_{0.08}Oₓ catalyst physically mixed with Pd/SiO₂ for ODH of ethane to acetic acid. The article claimed that precipitation in the presence of colloidal TiO₂ led to a tremendous increase in ethene and acetic acid rates (per active oxide) without significant changes in selectivity relative to unsupported samples. However, the conversion of ethane is lower than 5.5% which leads to a major portion of unconverted ethane needing to be separated and recycled back to the reactor system. The economic cost of the ethane recycle is very high.

It would be desirable to produce an improved catalyst which can selectively produce oxygenates from light alkanes with high productivity and stability in a single stage catalytic process. In addition, a process which integrates an advanced product separation technology is also important for commercialization.

Disclosed herein is a process that can include or comprise of a catalytic reaction system and a separation system. The catalytic reaction system can convert light alkanes such as ethane directly to acetic acid in the presence of oxygen and water. The separation system accepts the product effluents from the reaction system to separate acetic acid product from impurities, reaction byproducts, inert gases, and unconverted reactants and produce high purity acetic acid.

The catalytic reaction system can include one reactor or multiple reactors connected in series, converting at least a portion of the light alkanes directly to oxygenates in the presence of oxygen and water. The oxygen containing gas and water can be introduced at either the inlet of the first reactor or introduced at each inlet of the reactors when multiple reactors are used. The form of oxygen in the oxygen containing gas can be either as pure oxygen, as oxygen present in air, or as an oxygen enriched stream. An oxygen enriched stream refers to any stream having an oxygen concentration greater than the atmospheric concentration of oxygen. The oxygen stream can be obtained at a desired purity from an oxygen storage tank, or via an oxygen enrichment process, for example, the separation of air into nitrogen and oxygen, such as pressure swing adsorption (PSA), vacuum swing adsorption (VSA), or cryogenic separation techniques. In some aspects, the oxygen concentration in the oxygen containing gas may have at least about 70 vol %, at least 80 vol %, or at least 90 vol % oxygen (e.g., 90, 91, 92, 93, 94, 95, 96, 97, 98, 99, 99.1, 99.2, 99.3, 99.4, 99.5, 99.6, 99.7, 99.8, 99.9, or 100 vol % oxygen).

Within the reactor system, one or multiple supported catalysts can be used. When more than one catalyst is used, the catalysts can be uniformly mixed prior to the catalyst loading; in some embodiments, the different catalyst can be loaded separately as stacked bed within one reactor, or separately loaded in different reactors.

In some embodiments, only one catalyst is used in the reactors. The catalyst can contain molybdenum, vanadium, niobium, titanium, precious metals, and/or oxides thereof. The oxides of molybdenum, vanadium, and niobium can form a molybdenum-niobium-vanadium oxide crystallite. Precious metals may be amorphously well dispersed. The titania may have an anatase and a rutile crystallite structure. One exemplary support is titania, though others may be used as well.

In some embodiments, only one catalyst is used in the catalytic reactors. The catalyst can contain the oxides of molybdenum, vanadium, niobium, cerium, titanium, and precious metals. The oxides of molybdenum, vanadium, and niobium forms a molybdenum-niobium-vanadium oxide crystallite. Precious metals may be amorphously well dispersed. One exemplary support is a mixture of ceria and titania.

In some embodiments, only one catalyst may be used in the catalytic reactors. The catalyst can contain the oxides of molybdenum, vanadium, niobium, cerium, titanium, zirconium, and precious metals. The oxides of molybdenum, vanadium, and niobium can form a molybdenum-niobium-vanadium oxide crystallite. Precious metals may be amorphously well dispersed. One exemplary support is a mixture of ceria, titania, and zirconia.

In some embodiments, only one catalyst may be used in the catalytic reactors. The catalyst comprises the oxides of molybdenum, vanadium, niobium, cerium, zirconium, and precious metals. The oxides of molybdenum, vanadium, and niobium forms a molybdenum-niobium-vanadium oxide crystallite. Precious metals may be amorphously well dispersed. One exemplary support is a mixture of ceria and zirconia.

In some embodiments, two catalysts are used in the reactors. At least one catalyst can contain oxides of molybdenum, vanadium, niobium, and titanium. The oxides of molybdenum, vanadium, and niobium can form a molybdenum-niobium-vanadium oxide crystallite. Titania works as a support for the catalyst. Another catalyst comprises precious metals. The support for the catalyst can include titania, silica, alumina, and the combination thereof.

In some embodiments, two catalysts can be used in the reactors. At least one catalyst comprises the oxides of molybdenum, vanadium, niobium, cerium, and titanium. The oxides of molybdenum, vanadium, and niobium forms molybdenum-niobium-vanadium oxide crystallite.

The mixtures of ceria and titania serve as a support. Another catalyst can contain precious metals. The support for the catalyst including titania, silica, alumina, and the combination thereof.

In some embodiments, two catalysts can be used in the reactors. At least one catalyst can comprises oxides of molybdenum, vanadium, niobium, cerium, titanium, and/or zirconium. The oxides of molybdenum, vanadium, and niobium forms molybdenum-niobium-vanadium oxide crystallite. The mixtures of ceria, titania, and zirconia can serve as a support. Another catalyst can contain precious metals. The support for the catalyst including titania, silica, alumina, or any combination thereof.

In some embodiments, two catalysts can be used in the reactors. At least one catalyst can contain oxides of molybdenum, vanadium, niobium, cerium, zirconium, and/or oxides thereof. The oxides of molybdenum, vanadium, and niobium can form molybdenum-niobium-vanadium oxide crystallite. The mixtures of ceria and zirconia serve as a support. Another catalyst comprises precious metals. The support for the catalyst including titania, silica, alumina, and the combination thereof.

The reactor type can include any one of a fixed bed reactor, CSTR, fluidized bed reactor, moving bed reactor, CCR reactor, or the combination thereof. This disclosure is directed to the process of oxidative dehydrogenation of light alkanes, using catalytic materials such as oxides of vanadium, molybdenum, niobium, cerium, titanium, zirconium, and precious metal and the like as catalysts and molecular oxygen, O₂, as an oxidant in the presence of water (e.g., as steam) along with separation techniques to enable continuous production of oxygenates products. This disclosure includes reactor alternatives, catalyst alternatives, and reactants and products separation process alternatives.

Although not wanting to be bound by theory, the reactions involved in this process can be listed in the following equations:

$${C}_{2} {H}_{6} + \frac{1}{2} {O}_{2} \to {C}_{2} {H}_{4} + {H}_{2} O \qquad ( Eq . 1 )$$

$${C}_{2} {H}_{4} + {H}_{2} O \to {C}_{2} {H}_{5} OH \qquad ( Eq . 2 )$$

$${C}_{2} {H}_{5} OH + \frac{1}{2} {O}_{2} \to {CH}_{3} CHO + {H}_{2} O \qquad ( Eq . 3 )$$

$${CH}_{3} CHO + \frac{1}{2} {O}_{2} \to {CH}_{3} COOH \qquad ( Eq . 4 )$$

$${C}_{2} {H}_{6} + \frac{7}{2} {O}_{2} \to 2 {CO}_{2} + 3 H 2 O \qquad ( Eq . 5 )$$

The reaction selectivity toward a particular product can be somewhat controlled by the process conditions and catalysts, particularly for making ethylene, acetic acid, and CO₂. Typically, only trace amount of the ethanol and acetaldehyde are formed in the process due to further oxidation. The catalyst containing precious metal promotes the production of acetic acid, particularly in the presence of water. The higher reaction temperature and higher oxygen to ethane ratio tend to make more CO₂ through complete oxidation.

The catalyst used in the process may be important for obtaining sufficient yields. Adding niobium to an MoVO system can help improve the dispersion of MoVO system. This results in MoVNbOₓ system with better lower temperature activity and better stability. But the ethylene selectivity of this catalytic system is still higher than acetic acid selectivity. The much higher acetic acid selectivity can be achieved by adding precious metals onto the MoVNbOₓ system either through an impregnation of precious metal precursors to the MoVNbOₓ oxides, or physically mixing the supported precious metal catalysts with MoVNbOₓ oxide. However, this type of bulk catalyst typically has relatively low surface area which leads to low acetic acid productivity. In some aspects, titania with high surface area may be used as support for MoVNbOₓ. Titania (TiO₂) has many favourable properties that make it a good support due its non-toxicity, chemical stability and relatively low cost compared with other catalysts. In addition, titania itself is good catalyst which can oxidize directly or indirectly a wide range of chemical species. One catalyst preparation adds titania to the solution of NH₄VO₃ (ammonium metavanadate) and (NH₄)₆Mo₇O₂₄·4H₂O (ammonium heptamolybdate tetrahydrate) firstly and then adds a solution of C₄O₈NbOH·NH₃ (ammonium niobate (V)) oxalate hydrate to the above suspension. In this way, niobium oxide stays isolated from MoVOₓ or only small portion of niobium oxide embedded into the crystal structure of MoVOₓ. Therefore, the function of NbOₓ for enhancing the dispersion of MoVOₓ oxide may not be as effective. The concept of precursor of active sites is used in this disclosure, i.e., the complex of active sites is formed firstly prior to depositing them on the support. In this way, the active sites with optimum composition and uniform distribution will be formed largely on the final catalyst. The ensemble of active site precursor can be formed by mixing all compounds containing active elements at ambient temperature and atmospheric pressure, or at elevated temperature (heated condition), or at a system with elevated temperature and pressure such as hydrothermal synthesis condition. Afterwards, the mixture containing active site precursor can be applied onto the support. The catalyst made in this method will generally have improved dispersion, enhanced activity, and better stability. The active site precursor concept can be applied to one function of active site, or multiple functions of active sites depends on how the active elements are grouped together.

Cerium can be a good oxygen storage material due to its easy transformation between Ce⁴⁺ and Ce³⁺ species. When ceria, titania and zirconia work together as oxygen storage material, its oxygen storage capacity (OSC) is greatly improved compared to ceria alone. This feature might be helpful for the formation of active lattice oxide ions. Many studies have been made in this area which suggested the important role of lattice oxide ions in the selective oxidation of hydrocarbons over metal oxide catalysts.

The active elements include molybdenum, vanadium, niobium, cerium, titanium, zirconium, and precious metals. There are several alternatives which can make them work for converting ethane selectively toward acetic acid:
(1) One catalyst is used in the reactors: MoVNbOₓ group together as one type of active site; precious metal(s) play different role as another type of active site. When titania is used as a support, after the addition sequence and heat treatments, this type of catalyst can be denoted as PG/MoVNbOₓ/TiO₂ (PG=Precious Metal Group, the molar ratio of each element is not defined in the expression).
(2) One catalyst is used in the reactors: MoVNbOₓ group together as one type of active site; precious metal(s) play different role as another type of active site. When ceria and titania are used as a support, after the addition sequence and heat treatments, this type of catalyst can be denoted as PG/MoVNbOₓ/CeTiO_{y} (PG=Precious Metal Group, the molar ratio of each element is not defined in the expression).
(3) One catalyst is used in the reactors: MoVNbOₓ group together as one type of active site; precious metal(s) play different role as another type of active site. When ceria, titania, and zirconia are used as a support, after the addition sequence and heat treatments, this type of catalyst can be denoted as PG/MoVNbOₓ/CeTiZrO_{y} (PG=Precious Metal Group, the molar ratio of each element is not defined in the expression).
(4) One catalyst is used in the reactors: MoVNbOₓ group together as one type of active site; precious metal(s) play different role as another type of active site. When ceria, titania, and zirconia are used as support, after the addition sequence and heat treatments, this type of catalyst can be denoted as PG/MoVNbOₓ/CeZrO_{y} (PG=Precious Metal Group, the molar ratio of each element is not defined in the expression).
(5) Two catalysts are used in the reactors: MoVNbOₓ group together as one type of active site. Titania is used as support, and after the addition sequence and heat treatments, this type of catalyst can be denoted as MoVNbOₓ/TiO₂. Another catalyst contains precious metal(s) as active components. The support for the catalyst includes titania, silica, alumina, and the combination thereof. This type of catalyst can be denoted as PG/MO_{z}. (PG=Precious Metal Group, MO_{z}=titania, silica, alumina, and the combination thereof).
(6) Two catalysts are used in the reactors: MoVNbOₓ group together as one type of active site. Ceria and titania are used as a support, and after the addition sequence and heat treatments, this type of catalyst can be denoted as MoVNbOₓ/CeTiO_{y}. Another catalyst contains precious metal(s) as active components. The support for the catalyst includes titania, silica, alumina, and the combination thereof. This type of catalyst can be denoted as PG/MO_{z}. (PG=Precious Metal Group, MO_{z}=titania, silica, alumina, and the combination thereof).
(7) Two catalysts are used in the reactors: MoVNbOₓ group together as one type of active site. Ceria, titania, and zirconia are used as a support, after the addition sequence and heat treatments, this type of catalyst can be denoted as MoVNbOₓ/CeTiZrO_{y}. Another catalyst contains precious metal(s) as active components. The support for the catalyst includes titania, silica, alumina, and the combination thereof. This type of catalyst can be denoted as PG/MO_{z}. (PG=Precious Metal Group, MO_{z}=titania, silica, alumina, and the combination thereof).
(8) Two catalysts are used in the reactors: MoVNbOₓ group together as one type of active site. Ceria and zirconia are used as a support, after the addition sequence and heat treatments, this type of catalyst can be denoted as MoVNbOₓ/CeZrO_{y}. Another catalyst contains precious metal(s) as active components. The support for the catalyst includes titania, silica, alumina, and the combination thereof. This type of catalyst can be denoted as PG/MO_{z}. (PG=Precious Metal Group, MO_{z}=titania, silica, alumina, and the combination thereof).

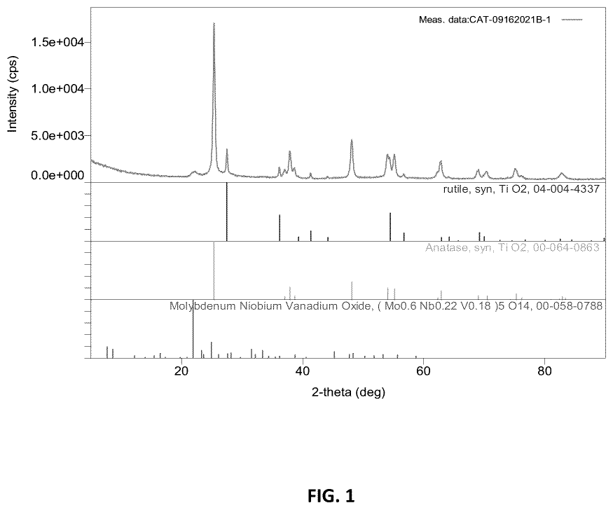

The X-ray diffraction results of one catalyst is depicted in FIG. 1. The sample used for the characterization is the catalyst MoVNbOₓ/TiO₂ without containing any precious metals. As shown in the XRD pattern, anatase and rutile are two dominant crystallites. The peak at 22 degree of 2theta is belonged to the crystallite of Molybdenum Niobium Vanadium Oxide (Mo_{0.6} Nb_{0.22}V_{0.18})₅O₁₄. The crystallite size for the sample is less than about 100 angstroms, less than about 90 angstroms, less than about 80 angstroms, less than about 75 angstroms, at least about 65 angstroms, at least about 70 angstroms, or about 73±4 angstroms based on the peak. In one exemplary embodiment, the crystallite size is about 65 angstrom to about 80 angstrom or about 69 angstrom to about 77 angstrom based on the peak. Compared to the samples made via other methods, the crystallite size is smaller, and can provide better dispersion for the oxides made by this new method.

The loadings of total molybdenum, vanadium, niobium should be greater than about 10 wt % of the total weight of the catalyst, greater than about 20 wt % of the total weight of the catalyst, or greater than about 30 wt % of the total weight of the catalyst. The catalyst should comprise at least about 70 wt %, at least about 80 wt %, or at least about 90 wt % support based on the total weight of the catalyst.

In some embodiments, for better utilization of precious metal, the precursors of precious metal including one of ruthenium (Ru), rhodium (Rh), palladium (Pd), osmium (Os), iridium (Ir), platinum (Pt), gold (Au), and any combination thereof, are added on the thermally treated oxides of molybdenum, vanadium, niobium, cerium, titanium, and zirconium to form a single catalyst. The total loading of the precious metal is less than about 1 wt %, less than about 0.1 wt %, or less than about 0.05 wt % based on the total weight of the catalyst.

In some embodiments, for better utilization of precious metal, the precursors of precious metal including one of ruthenium (Ru), rhodium (Rh), palladium (Pd), osmium (Os), iridium (Ir), platinum (Pt), gold (Au), and any combination thereof, are added on the titania, silica, and alumina and the combination thereof to form another catalyst. The total loading of the precious metal is less than about 1 wt %, less than about 0.3 wt %, or less than about 0.05 wt % based on the total weight of the second catalyst.

When the oxides of molybdenum, vanadium, niobium, cerium, titanium, precious metals are existing in two separate catalysts such as MoVNbOₓ/TiO₂ and supported precious metal catalyst, they can be loaded to the reactor as a physical mixture, or loaded separately in stacked bed, or loaded in separate reactors.

The reactor type includes any one of a fixed bed reactor, CSTR, fluidized bed reactor, moving bed reactor, CCR reactor, or the combination thereof. The selection of reactor type is determined by the catalyst activity and the mechanism of the catalyst deactivation.

In one embodiment of the laboratory scale catalyst test, the catalyst performance test is carried out with a tubular fixed bed reactor. It is surrounded by brass block, in turn surrounded by a band heater. Reactor temperature is measured by an internal thermocouple that located in the center of the reactor.

Catalyst loading is as follows: glass wool is applied to the bottom of the reactor, followed by adding 4-millimeter (mm) size of glass beads and then adding 1 mm size of glass beads. All the glass beads are treated with a mineral acid (e.g., 5% nitric acid) and rinsed with DI-water until the pH is near 7. The catalyst can be loaded with or without diluting with inserts such as glass beads, quartz chips, or silicon carbide (SiC). The catalyst is added into the reactor with or without mixing the inert followed by adding smaller size of glass beads, larger size of glass beads and glass wool to form the catalyst bed. The center of catalyst bed is ensured to align with the tip of the internal thermal couple.

After the reactor is connected to the test unit, the pressure check is implemented with nitrogen. To avoid the ethane concentration exceeding the upper limit of ethane flammability, the system is purged with nitrogen to ensure an oxygen free system. The reactor temperature is setup to the target temperature with the actual temperature control at target temperature±1° C. An ethane flow is first established at a target flow and then an air flow is established at a target flow. A high pressure liquid chromatography (HPLC) pump is used to introduce a target amount of type I water per ASTM D1193-99e1 standard from the reactor inlet. The pressure reactor system is controlled in the range of 100 psi (7 bar) to 3,000 psi (200 bar) via a back pressure regulator.

The liquid product is obtained via a condenser and collected routinely for gas chromatograph (GC) analysis. The vent gas is also collected at atmospheric pressure (atm) for GC analysis. The calculations of test results are based on the GC analytical results. The calculations of the catalyst performance are indicated as the following equations:

$$Ethane Conversion ( \% ) = \frac{( Ethane Flow IN - E thane Flow OUT )}{E thane Flow IN} \times 100$$

$$Acetic Acid Selectivity ( \% ) = \frac{Moles of Acetic Acid Prodcued}{( Moles of Ethane IN - Moles of Ethane OUT )} \times 100$$

$${CO}_{2} Selectivity ( \% ) = \frac{Molar of {CO}_{2} Prodcued}{( Moles of Ethane IN - Moles of Ethane OUT )} \times 100$$

$$Acetic Acid Productivity ( g / kg cat . h ) \frac{Grams of Acetic Acid Produced per Hour}{Kilograms of Catalyst Loaded} \times 100$$

The reaction temperatures are in the range of about 150° C. to about 600° C., in the range of about 240° C. to about 500° C., or in the range of about 260° C. to about 450° C.

The reaction pressures are in the range of about 50 psi (3 bar) to about 3,000 psi (200 bar), in the range of about 100 psi (7 bar) to about 2500 psi (170 bar), or in the range of about 200 psi (10 bar) to about 2,000 psi (100 bar).

The gas hourly space velocity is in the range of about 50 h⁻¹ to about 20,000 h⁻¹, in the range of about 100 h⁻¹ to about 15,000 h⁻¹, or in the range of about 200 h⁻¹ to about 10,000 h⁻¹.

The ethane concentration in the gas feed is greater than about 15%, greater than about 25%, or greater than about 35%.

The molar ratio of water addition to ethane is in the range of about 1/50 to about 10/1, in the range of about 1/25 to about 5/1, or in the range about 1/15 to about 3/1.

The ethane conversion in a single path can be higher than about 3%, higher than about 5%, higher than about 7.5%, or higher than about 10%. In some aspects, the ethane conversion in a single path can be higher than about 20%, higher than about 30%, or higher than about 40%.

The acetic acid selectivity should be higher than about 30 mol %, should be higher than about 45 mol %, or should be higher than about 60 mol %.

The acetic acid productivity should be higher than about 50 g/kg cat.h, should be higher than about 75 g/kg cat.h, should be higher than about 100 g/kg cat.h, or should be higher than about 150 g/kg cat.h after 100 hours of time on stream.

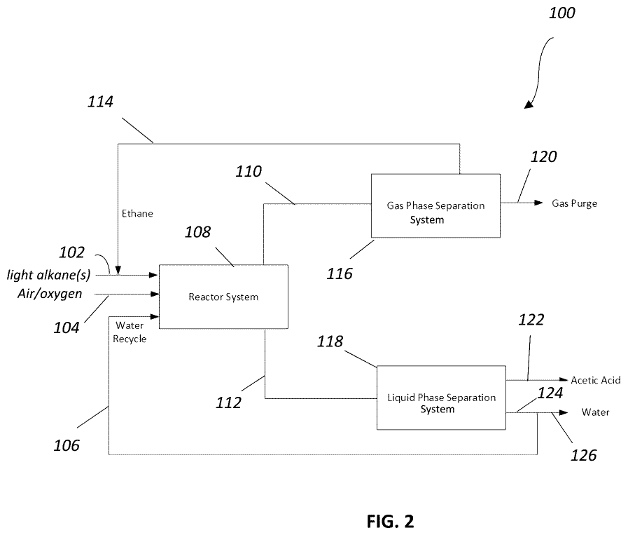

An exemplary overall process flow diagram is shown in FIG. 2. A catalytic processing apparatus 100 for manufacturing acetic acid comprises three main sections: a reactor system 108, a gas phase separation system 116, and a liquid phase separation system 118. Feeds of a hydrocarbon in stream 102 such as ethane, optionally combined with recycled fluids as discussed hereinafter, and air 104 are provided to the reactor system 108. The reactor system 108 provides at least a primarily gas effluent 110 to the gas separation system 116. The effluent 110, at least substantially vapor, leaving the reactor system 108 comprises unreacted ethane, non-condensable nitrogen, unreacted oxygen, byproduct carbon dioxide, and small amount of acetic acid, water and other reaction byproducts. Effluent 110 is provided to the gas separation system 116 which provides a recycle stream 114 and a purge stream 120. Depending on the hydrocarbon content, composition and the heat value, the purged stream may be used as fuel gas for generating water and/or electricity. A subset of this case would be sending all the unreacted gas stream to a furnace. The recycle stream, after compression, can be combined with ethane stream 102 and provided to the reactor system 108. The gas separation system may comprise a membrane system, a pressure swing adsorption system, an absorber, etc. Alternatively, effluent 110 may simply be split into recycle stream 114 and gas purge stream 120.

The primarily liquid stream 112 from the reactor system 108 comprises acetic acid, water, ethanol, methanol, acetaldehyde, acetone, methyl acetate, isopropanol, ethyl acetate, formic acid, other reaction by-products, and small amounts of dissolved gases such as nitrogen, oxygen, ethane, and carbon dioxide. Acetic acid has a higher boiling point (118° C.) than other by-products such as ethanol (78.2° C.), acetaldehyde (20.2° C.), methanol (64.5° C.), acetone (56° C.), methyl acetate (57.1° C.), isopropanol (82.5° C.), ethyl acetate (77.1° C.), and water (100° C.). Some of the by-product impurities (ethanol, ethyl acetate) form minimum-boiling azeotropes with water, whereas acetic acid forms a tangent pinch with water. Dissolved gases can be recovered using the liquid phase separation system 118 which may include at least one of a flash vessel, a gas vent on downstream distillation columns, and a degassing column. Any by-products heavier than acetic acid can be removed by adding a second distillation column, where product acetic acid is removed as distillate and heavy impurities are removed as the bottoms of the second column. The liquid phase separation system 118 can provide an acetic acid product stream 122, and a stream 124 including at least substantially water, although some acetic acid, feed impurities, reaction by-products, and reaction intermediates may be present. A portion 106 of the stream 124 can be a recycle stream 106 provided to the reactor system 108 with remainder being purged via a stream 126. Further details of the process streams and systems are provided with respect to various embodiments discussed below.

Oxidizing ethane to acetic acid is an exothermic reaction, and the heat generated in the reactor is removed to keep the temperature in the reactor within the desired range. The reactor system 108 can be configured to be cooled in-situ or operated as adiabatic reactor with external cooling. In-situ cooling may allow the reactor to operate as an isothermal reactor or a substantially isothermal reactor, though the reactor operating temperature may be controlled using an in-situ coolant without operating in an isothermal regime (e.g., by having a controlled or target temperature rise across the reactor). For in-situ cooling, heat effects of the reaction can also be removed by means of an embedded cooling or heat removal mechanism. The presence of an inert gas, such as nitrogen, other recycled inert gases (e.g., CO₂), as well as recycled water can also be used to mitigate the temperature rise in the reactors.

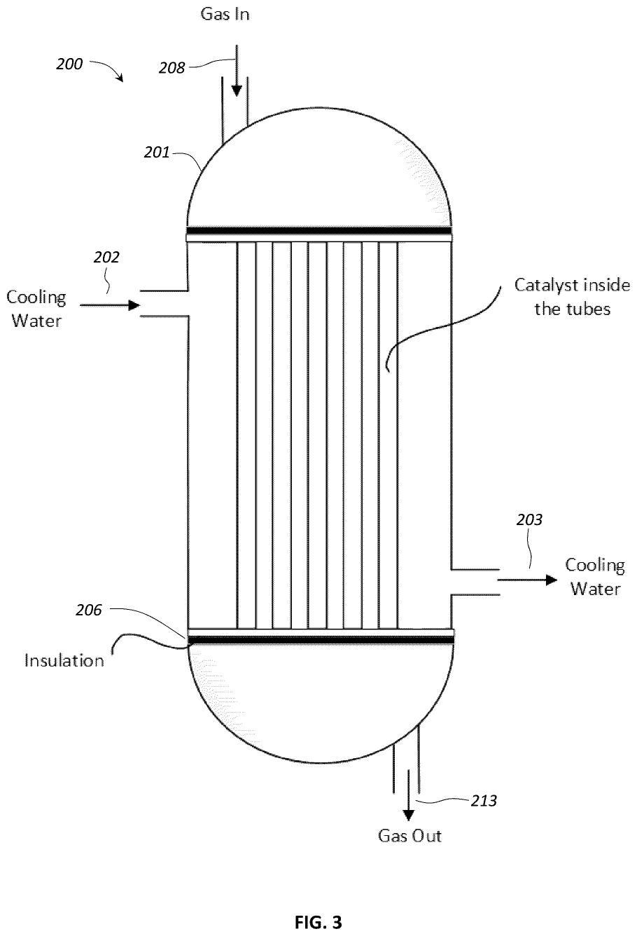

Referring to FIG. 3, the reactor system 108 may include a reactor 200, such as a shell-and-tube reactor 200. Generally, this isothermal configuration continuously maintains the heat of reaction using a heat transfer fluid. The heat transfer fluid can be external to the process (e.g., boiler feed water which could be used to generate steam for use in the process) and/or a cold stream in the process (e.g., cold feed or recycle streams). Heat can be transferred from the reactor to the coolant using a cooling jacket around the reactor/catalyst, or tubes/coils embedded with the reactor. A common configuration is a shell-and-tube reactor 200, with the catalyst either in the tubes or on the shell side. The preferred reaction temperature is between about 250° C. and about 375° C. and preferred pressure of about 50 psi (3 bar)—about 500 psi (30 bar). The reactor outlet can be cooled and phase separated to yield a vapor and liquid product.

Referring back to FIG. 3, a shell/tube configuration allows heat transfer for removal of the heat of reaction within the reactor vessel 201. The reactant fluids (e.g., gases), can enter the reactor in stream 208, and any unreacted gases can pass out of the reactor as stream 213. Heat transfer fluid (e.g., water, an aqueous fluid, glycol, an oil, etc.) can be used to maintain the temperature in the reactor at a desired temperature. The heat transfer fluid can be introduced in stream 202 and pass out of the reactor vessel 201 as stream 203. The reactor vessel 201 can have insulation 206 disposed on a portion or all of the reactor to maintain the temperature at a desired set point. While shown in FIG. 3 as having the reactants and catalyst on the tube side and the heat transfer fluid on the shell side of the exchanger, the reactants and catalyst can also be introduced on the shell side and the heat transfer fluid can be within the tubes in some embodiments.

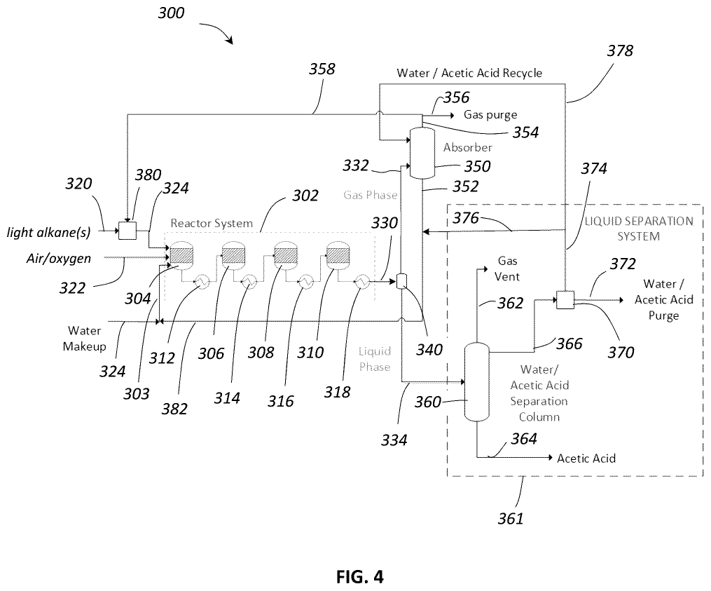

Referring to FIG. 4, the reactor system 108 may be a reactor system 302 including an adiabatic cascade, in a catalytic processing apparatus 300, which includes the reactor system 302 and a liquid separation system 361. This configuration comprises a cascade of adiabatic stages or reactors 304, 306, 308, and 310 with exchangers or coolers 312, 314, 316, and 318 between or after each of the stages or reactors 304, 306, 308, and 310. The hydrocarbon feed stream 320, optionally combined with a gaseous recycle stream 358, discussed below, and air stream 322 are provided to each reactor via the first reactor 304 or to each reactor 304, 306, 308, and 310 individually in the cascade. The feed to each reactor can comprise the cooled reactor effluent from the previous reactor in the cascade, and/or air and/or ethane and/or recycled water (vaporized). Each adiabatic reactor 304, 306, 308, and 310 in the cascade operates with a temperature rise of approximately of 50° C. (for example, from 275° C. to 325° C.). The temperature rise can be controlled by varying the flowrate of air and/or ethane to each reactor 304, 306, 308, and 310. The reactor outlet from each reactor 304, 306, 308, and 310 in the cascade is cooled to the desired inlet temperature of the next reactor in the cascade using either a cooler and/or direct contact with the cold feed to next reactor in the cascade. As in the isothermal configuration, an external coolant or a cold process stream can be used in the cooler. Moreover, both recycled carbon dioxide and nitrogen, as discussed hereinafter, can act as diluents to mitigate the temperature rise in the adiabatic reactors, such that fewer cascade of reactors with intercoolers are required to achieve the desired conversion. Preliminary calculations indicate that a cascade with four stages can be used to achieve an overall conversion of 20%.

The effluent 330 from the last reactor 310 passes through a last cooler 318 to a flash drum 340. The flash drum 340 provides a gas stream 332 and a liquid stream 334. The gas stream 332 includes, in addition to nitrogen, byproduct carbon dioxide, unreacted ethane, and unreacted oxygen, acetic acid and carry over reaction byproducts. Recovering these reaction products can reduce product loss. The gas stream 332 is sent to an absorber 350. Absorption can be used to recover acetic acid and reaction by-products from the gas stream 332. Water or water containing small amounts of acetic acid can be used as a solvent in the absorber. A water and acetic acid recycle stream 378 can provide the solvent to recover acetic acid and other desirable compounds as an absorber liquid effluent 352, rich in acetic acid, and be combined with a stream 376 for providing a recycle stream 382 to the reactor system 302, as hereinafter described. Alternatively, the absorber liquid effluent 352 can be sent to a liquid separation system 361. The absorber 350 can be placed on either stream 332 to improve the per pass acetic acid recovery or on the purged gas stream 356 to reduce acetic acid loss.

The gas effluent 354 leaving the absorber 350 can be compressed and recycled as the stream 358 and combined with the ethane feed stream 320. The combined stream 324 can be provided to the reactor system 302. A portion of the gas effluent 356 can be purged to eliminate the build-up of undesirable compounds, and be sent to any desirable destination such as a flare.

The liquid stream 334 can be sent to the liquid separation system 361, for example, a single distillation column 360. The distillation column 360 can be used to recover high purity acetic acid from the liquid stream 334 leaving the reactor system 302. Acetic acid is recovered as a bottoms product 364 from the distillation column 360, while a gas stream 362 is vented. The distillation column 360 also produces a distillate 366 including water and the other light components. While it is theoretically possible to achieve a high acetic acid recovery (>99.5%, by weight), due to the presence of the tangent pinch this is usually not practical or economical, and the distillate will contain some acetic acid. Because water is produced in this process, a fraction of the water rich distillate 366 (which contains ˜5% percent, by weight, acetic acid) can be split in splitter 370 and purged via a stream 372 from the process and possibly sent to waste treatment, while the rest may be recycled as a stream 376 to the reactor system 302.

A fraction, or the entirety, of the stream 374 from the water separation column 360 can be used as the solvent in the absorber 350 provided via the stream 378. The acetic acid rich liquid stream 352 leaving the absorber 350 can be recycled to the reactor system 302 as described above, or the liquid separation system 361 or both. The conditions in the absorber 350 are the pressure and temperature of the reactor exit stream 330. The purge gas stream 356 is can be used as a fuel gas as mentioned above. The other portion of the stream 374 can be a stream 376 combined with the absorber liquid effluent 352 to form the recycle stream 382. The recycle stream 382 can be combined with a stream 324 providing make-up water to form a stream 303 provided to the reactor system 302.

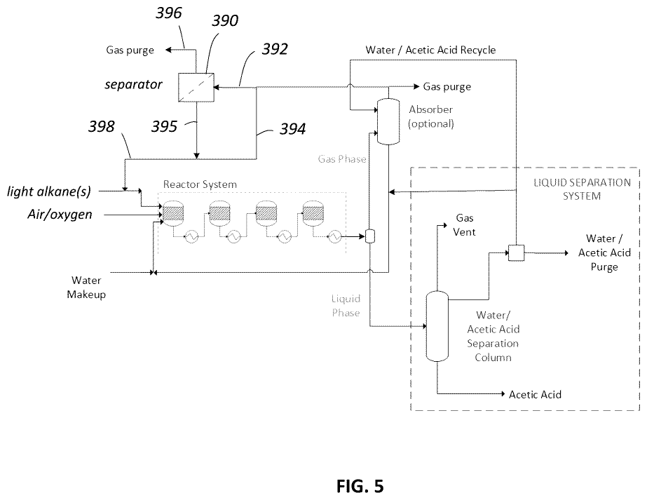

Referring to FIG. 5, another catalytic processing apparatus is depicted. This apparatus is similar to the apparatus depicted in FIG. 4, and like components, such as the reactor system, will not be re-described in the interest of brevity. A fraction 394 of this gas stream from the absorber is recycled, after compression, to the reactor system, while the remainder 392 is sent to a separation system e.g., a membrane system, pressure swing adsorption, etc. In this embodiment, the remainder is sent to a separator 390 such as a membrane assembly and/or a carbon dioxide scrubber where nitrogen, oxygen, and carbon dioxide are separated from other components in the gas stream, and purged via stream 396 from the process. The hydrocarbon rich stream 395 is subsequently recycled by combining with the fraction 394 to form a stream 398 sent to the reactor system. A subset of this would be the case in which the entire gas stream is sent to the separation system. In either instance, an additional purge stream can be used to prevent build-up of other gaseous impurities.

The system of FIG. 5 can also be used with an oxygen enhanced stream or pure oxygen as the feed stream. In some embodiments, the oxygen component can be replaced by an enhanced oxygen stream having a purity ranging from about 95 mol % to about 99.99 mol %. As shown, a fraction of the gas stream from the absorber (e.g., stream 392) can be sent to a separator 390. When oxygen is used as the input, the separator 390 may be a carbon dioxide separation system such as a caustic scrubber, an amine-based extraction and recovery, or the like to provide for the removal of carbon dioxide produced in the reactors. A purge stream from the absorber and/or a purge stream 396 from the separator 390 can be used to prevent build-up of other gaseous impurities. In this embodiment, the scrubbed gas stream 395 now containing mainly unreacted hydrocarbon and trace amounts of oxygen, nitrogen and carbon dioxide, can be sent back to the reactor system. The scrubbed gas stream 395 can be recycled and combined with the fraction 391 along with additional ethane, oxygen, and water to be sent to the reactor system. In some embodiments, all of the gas stream from the absorber can pass to the separator 390 such that no gas passes through stream 394.

Azeotropic distillation uses an entrainer to separate two components that are difficult to separate by conventional distillation, either due to the presence of an azeotrope or tangent pinch behavior. Entrainers in the separation of acetic acid and water can include components such as ethyl acetate, propyl acetate, butyl acetate, etc. The salient characteristic of an entrainer is that the entrainer forms a minimum boiling heterogeneous azeotrope.

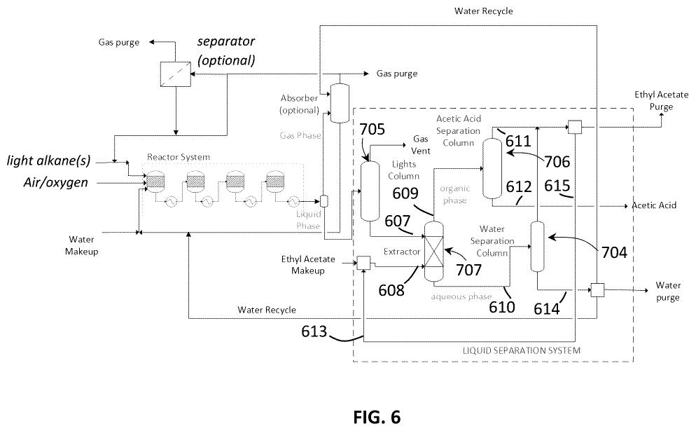

Referring to FIG. 6, another catalytic processing apparatus is depicted. The reactor system of this apparatus is similar to the apparatus depicted in FIG. 4, and like components, such as the reactor system, will not be re-described in the interest of brevity. In the liquid separation system, an entrainer such as ethyl acetate can be used to separate water and acetic acid to yield two high purity products. The liquid product from the reactor system first goes to a lights column 705, where methanol, ethanol, acetaldehydes, acetone and other light impurities are recovered, in order to prevent them from accumulating in the downstream recycle loops and thereby minimizing the entrainer purge. The bottoms from the lights column 705 is provided to an extraction column 707, which may be a multi-stage column or a simple decanter (i.e., a single-stage extraction column). An entrainer or solvent can be introduced in the lower portion of the extraction column 707 through stream 608, while the liquid stream 607 can be introduced in an upper portion of the extraction column 707. The solvent can rise within the extraction column 707 to extract at least a portion of the acetic acid in the solvent phase, which forms a heterogeneous mixture with the aqueous phase liquid in the liquid stream 610. More specifically in the extraction column 707, the acetic acid rich liquid stream can be combined with an entrainer rich stream to yield an organic stream comprising acetic acid and the entrainer, and an aqueous stream comprising water and the entrainer. The rich solvent containing the extracted acetic acid can then pass out through stream 609, while the aqueous phase having the acetic acid at least partially removed can pass out through liquid stream 610 While shown as having the liquid stream 607 entering the upper portion of the extraction column 707, the liquid stream 607 could enter a lower portion if the solvent has a higher density than the aqueous phase in the liquid stream 610, where the counter-current flow is established based on density differences between the two streams.

As shown in FIG. 6, the acetic acid rich solvent in stream 609 can then be sent on to an acetic acid separation column 706 where high purity acetic product is produces as the bottoms product 612. The solvent rich distillate 611 is recycled to the extractor column 707. A small purge can be taken out of the recycled solvent stream to avoid the build-up of impurities.

The water rich stream 610 can be provided to the water separation column 704, where water can be recovered as the bottoms stream 612 and the solvent rich distillate stream 615 is recycled to the reactor. Part of the water stream 614 can be purged from the process, and subsequently treated, if necessary, before being discharged, and the rest can be recycled to the reactor. Note that the purge in this case does not contain a significant amount of acetic acid. Consequently, the overall acetic acid recovery is higher than that for the separation sequences without an entrainer (e.g., FIG. 4), but at a cost of additional columns.

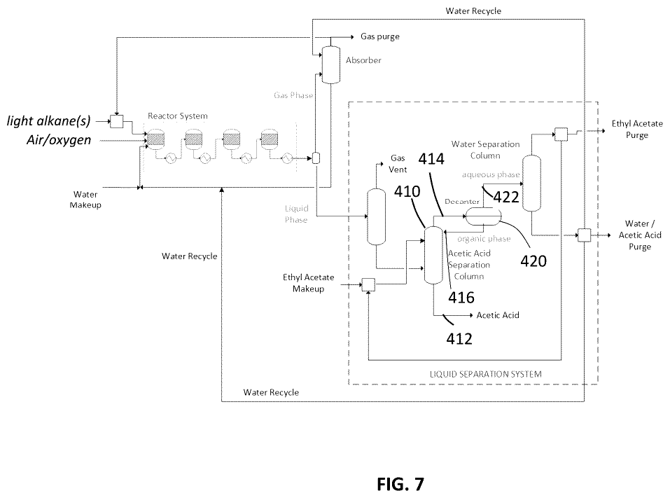

Referring to FIG. 7, yet another catalytic processing apparatus is depicted. This apparatus is similar to the apparatus depicted in FIG. 6, and like components, such as the reactor system, will not be re-described in the interest of brevity. This apparatus of FIG. 7 also uses an entrainer. The liquid product from the reactor system first goes to a lights column, where methanol, ethanol, acetaldehydes, acetone and other light impurities are recovered, in order to prevent them from accumulating in the downstream recycle loops and thereby minimizing the entrainer purge. A bottoms of the lights column is provided to the acetic acid separation column 410. The acetic acid separation column 410 produces a bottoms 412 including an acetic acid product. A tops 414 of the acetic acid separation column 410 includes water and ethyl acetate and is provided to a decanter 420. The water and ethyl acetate settle and separate with the ethyl acetate removed as stream 416 and returned to the acetic acid separation column 410. Stream 422 comprises water and is provided to the water separation column.

For example, any of the gas streams (e.g., stream 110, stream 332, stream 358, etc.) in FIGS. 2-7 can be sent to a separation system. The membranes can comprise organic or inorganic membranes that can be used to separate light hydrocarbons from nitrogen, hydrogen and oxygen.

In some embodiments, molecular sieves can be used to separate the components of the gas outlet streams. Pressure swing adsorption using molecular sieves or other materials such as titano-silicates can also be used as an effective method to separate light hydrocarbons from gases such as CO₂, nitrogen (N₂), and O₂. The gas outlet streams from the separator that follows the reactor can be passed through a bed of adsorbent media at high pressure. Under high pressure, the specific gasses can be selectively adsorbed on to the surface and pores of the adsorbent media. The adsorbed gases are removed from the molecular sieves by reducing the pressure. Commercial scale systems typically use multiple adsorbent beds so that one is always in the adsorption mode, while other beds are regenerated, thus enabling continuous operation. Molecular sieve media with the appropriate pore size is used such that it will preferably adsorb target molecules. Smaller molecules, such as CO₂, O₂, and N₂ are able to fit into the pores and be strongly adsorbed, while the light hydrocarbons such as methane, ethane, etc., are too large to fit in the pore and move downstream of the adsorbent bed. This results in effective separation of light hydrocarbons from CO₂, O₂, and N₂. Separated light hydrocarbons can then be recycled back to the reactor, with a small purge, as seen in FIGS. 4-7 and described in more detail herein.

While shown in FIGS. 4-7 as having adiabatic reactor cascades, the reactor cascades can be operated in an isothermal configuration in some embodiments, with intercoolers being optional or removed. Isothermal operation with regards to the cooling system can include any of the options disclosed herein such as in-situ cooling for the reactors.

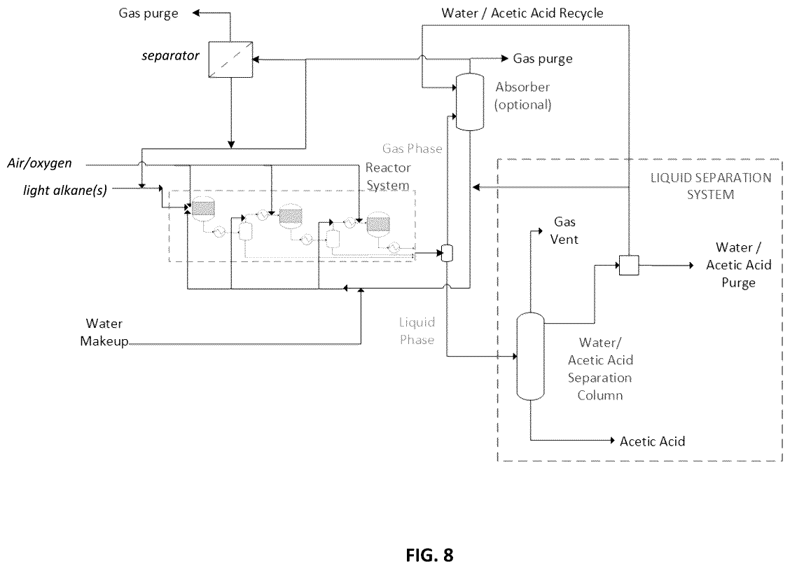

Another catalytic processing apparatus for the reactor cascade operating in an isothermal mode is depicted in FIG. 8. This apparatus is similar to the apparatus depicted in FIG. 5, and like components, such as the reactor system, will not be re-described in the interest of brevity. In this configuration, each reactor can be operated at a desired isothermal or substantially isothermal operating temperature by means of internal or external cooling as described herein. The reactor effluent from one or more of the reactors in the series can be cooled in a heat exchanger such that a condensed liquid product can be removed via a subsequent phase separator. The collected liquid phase from each stage can be sent to a liquid separation system, whereas the vapor phase can pass onto the next reaction stage after again being heated to the reaction temperature in a heater. The reactor effluent from the last stage can be cooled and phase separated, with the vapor phase going to the gas phase separation process as described with respect to FIG. 5. The benefit of having intercoolers with condensation for an isothermal process is to allow for a selectivity improvement by removal of product acetic acid from each stage. Makeup water can be added to the reactor feed after the phase separator, and this makeup water can be added in vapor form either before heating the vapor phase or after heating the vapor phase from the phase separator prior to its addition to the next reactor. In some aspects, the addition of steam can be used to provide at least a portion of the heat needed to reheat the gas stream passing to the next reactor.

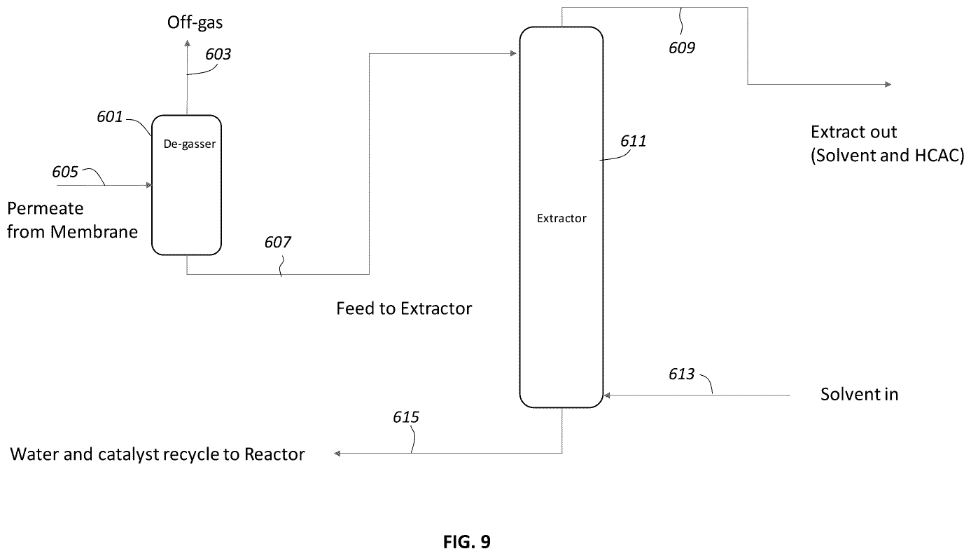

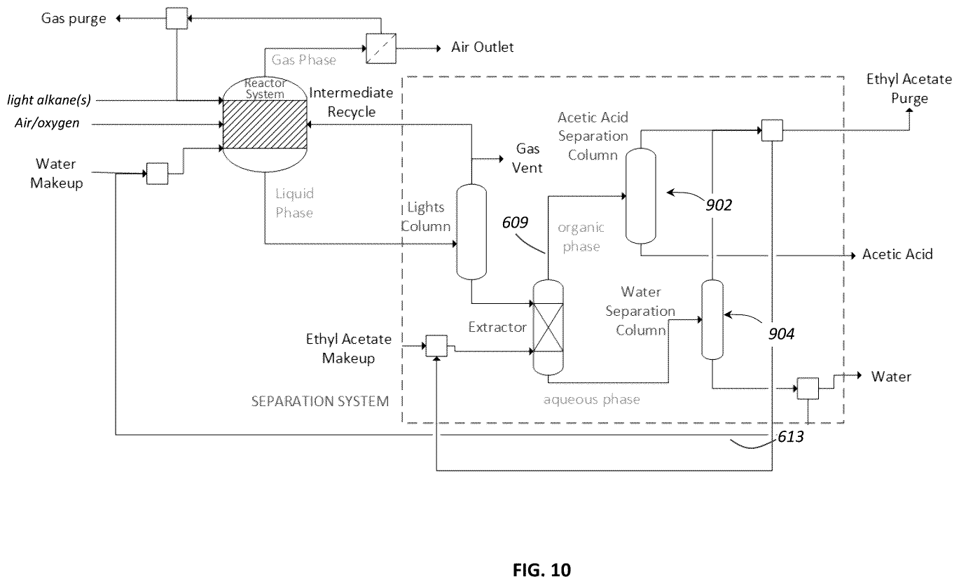

In some aspects, separation systems are shown in the schematic flowsheets of FIGS. 9 and 10. When the product comprises acetic acid, the objective of the separation system is to separate the desired chemical, acetic acid, from the liquid mixture that contains water, dissolved gases, some catalyst, intermediates, and other by-products.

As shown in FIG. 9, the product stream 605 from the reactor can first pass to a degassing unit 601. The liquid entering the degassing unit 601 can be degassed by using a distillation column or a simple flash vessel. In general, a flash vessel may have a single stage of separation where as a distillation column can comprise two or more stages of separation. Additional vessels or columns can be optionally used to further de-gas the product stream to a desired level. The degassing unit 601 can be used to vent out any dissolved gases such as N₂, O₂, hydrocarbon gases, hydrogen, or the like in the off-gas stream 603. The pressure and other operating conditions (e.g., temperature, flow rates, residence time, etc.) can be adjusted to reduce the loss of products in the vapor phase.

In some embodiments, the gas stream 603 vented from the product stream 605 in the degassing unit 601 can also comprise unreacted hydrocarbon gases exiting the reactor in the gas phase along with the non-condensable nitrogen and unreacted oxygen and carbon dioxide. The resulting gas stream can be processed in a number of ways. In some aspects, nitrogen can be separated from the unreacted hydrocarbon, and after an optional purge stream is taken from the recycle stream, the separated unreacted gas stream can then be compressed to the reactor pressure and recycled back to the reactor. In some aspects, a fraction of the unreacted hydrocarbon gas stream can be recycled to the reactor and the rest purged from the process gas without any type of membrane separation. Depending on the hydrocarbon content, composition, and heat value, the purged stream may be used as fuel gas for generating steam and/or electricity. In some aspects, all the unreacted gas stream can be sent for use as fuel in the process. The selection of the use of the recycle gas may depend on the economic of the system and can vary over time.

In some aspects, the vented gas stream (e.g., stream 110 in FIG. 2 or off gas stream 603 in FIG. 9) can be further treated to recover product prior to recycling and/or sending the vent stream for use as fuel. In addition to nitrogen, unreacted reactants, and possibly some unreacted oxygen, the vent gas stream can also comprise acetic acid and other reaction intermediates. In addition to the separation techniques described herein, the vent gas stream, or a portion thereof, can pass through an absorber or absorption unit. An absorption unity can comprise any unit configured to contact a gas stream with a solvent and absorb at least a portion of one component in the gas stream into the solvent, thereby effecting a separation of the components. Absorption can be used to recover acetic acid and reaction intermediates from the gas stream. The absorber can be placed on the purged gas stream (to reduce acetic acid loss) and/or the entire gas stream (to increase per pass acetic acid recovery). Water can be used as a solvent in the absorber. A fraction, or the entire, water recycle from the water separation column can be used as the solvent in the absorber. The acetic acid rich liquid leaving the absorber can be recycled to the reactor, or the liquid separation system or both. The conditions in the absorber can be approximately the same as the pressure and temperature of the reactor exit stream. The remaining purge gas stream can be used as a fuel gas within the system or leave the system.

The liquid stream 607 leaving the degassing unit 601 can comprise the product in an aqueous fluid. The liquid stream 607 can then pass to a separation unit 611 to produce an acetic acid stream and a second stream comprising other components such as water, any remaining catalyst, any gas solvating agents, and the like. The separation unit 611 can use any suitable separation techniques to separate from the acetic acid from the remaining components. In some aspects, the separation unit 611 can comprise an extractor that uses a solvent to extract the desired components. While shown as an extractor, in some embodiments, the separation unit can utilize distillation, decantation, azeotropic distillation, extraction, extractive distillation, or the like, and the separation unit 611 can be formed from one or more vessels connected in parallel or in series.

In some embodiments, distillation can be used to separate the acetic acid from the remaining components. However, the vapor-liquid behavior of acetic acid and water indicates the presence of a tangent pinch on the pure water side, which means that while it is possible to achieve a high purity acetic product using conventional distillation, it is difficult to simultaneously achieve a high purity water product. When some acetic acid is recycled along with the water back to the reactor, the desired separation can be achieved using conventional distillation

In some embodiments, azeotropic distillation can be used in order to address the tangent pinch. Azeotropic distillation uses an entrainer to separate two components that are difficult to separate by conventional distillation, either due to the presence of an azeotrope or tangent pinch behavior. Entrainers in the separation of acetic acid and water can include components such as ethyl acetate, propyl acetate, butyl acetate, etc. The salient characteristic of an entrainer is that the entrainer forms a minimum boiling heterogeneous azeotrope.

In some embodiments, decantation can be used to separate the acetic acid from the other components of the product stream. Decantation comprises the separation of acetic acid and water by introducing an entrainer that exhibits heterogeneous behavior with water. Acetic acid then distributes between the aqueous and organic phases. Decantation by itself is not able to obtain pure product purity desired. When an entrainer is used, the aqueous phase from the decanter can be further purified in a distillation column in which water is recovered as the bottom stream with the distillate recycled to the entrainer stream. The organic phase from the decanter can be further purified using distillation (e.g., in one or more columns) to recover high purity acetic acid, and the entrainer rich stream from the distillation may be recycled to the entrainer stream, after an optional purge.

Extraction followed by distillation is similar to decantation followed by distillation, and involves a liquid/liquid extraction step where an appropriate solvent suitable to form a multi-phase solution (e.g., at least a partially immiscible solvent as an extractive agent which exhibits heterogeneous behavior with water) is contacted in a counter-current fashion with the water based product stream. The solvent can be selected based on its physical properties so that it effectively and selectively extracts the acetic acid.

FIGS. 9 and 10 illustrate process flow configurations for the separation of the products from the reaction system. FIG. 9 shows an example of an extraction process in which the liquid stream 607 can be provided to an extraction column 611, which may be a multi-stage column. It can be noted that an extraction column can also serve as a decanter (e.g., a single-stage extraction column). An entrainer or solvent can be introduced in the lower portion of the extraction column 611 through stream 613, while the liquid stream 607 can be introduced in an upper portion of the column 611. The solvent can rise within the extraction column 611 to extract at least a portion of the acetic acid in the solvent phase, which forms a heterogeneous mixture with the aqueous phase liquid in the liquid stream 607. More specifically in the extraction column 611, the acetic acid rich liquid stream can be combined with an entrainer rich stream to yield an organic stream comprising acetic acid and the entrainer, and an aqueous stream comprising water and the entrainer. The rich solvent containing the extracted acetic acid can then pass out through stream 609, while the aqueous phase having the acetic acid at least partially removed can pass out through stream 615. The aqueous phase stream can contain the catalyst and can be recycled within the system to the reactor. While shown as having the liquid stream 607 entering the upper portion of the extraction column 611, the liquid stream 607 could enter a lower portion if the solvent has a higher density than the aqueous phase in the liquid stream 607, where the counter-current flow is established based on density differences between the two streams.

As shown in FIG. 10, the rich solvent in stream 609 can then be sent on to an acetic acid separation column 902 for separation of the acetic acid and the solvent. The selection of the solvent can be used to provide an easier separation than the separation of water and acetic acid. Once separated, the solvent phase from an extraction or decanter process can be partially recycled back to the solvent stream 613. A small purge can be taken out of the recycled solvent stream to avoid the build-up of impurities.

Water can be recovered in a distillation column 904 with water being removed as the bottoms stream and the distillate recycled to the solvent product from the acetic acid separation column 902. Part of the water can be purged from the process, and subsequently treated, if necessary, before being discharged, and the rest can be recycled back to the reactor.

The separation system may also take other components of the reaction mixture into account. Acetic acid production by oxidation of hydrocarbons as described herein may also produce intermediates, such as ethanol and acetaldehyde, and by-products, such as carbon dioxide. Both intermediates and by-products are partially recycled with the unreacted gases back to the reactor whereas a fraction is purged with the gas or liquid purge.

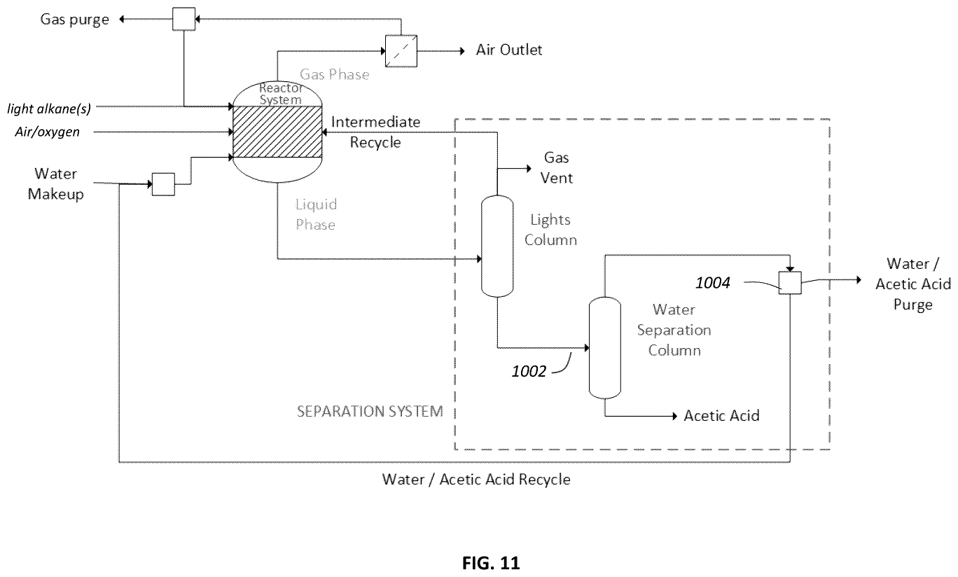

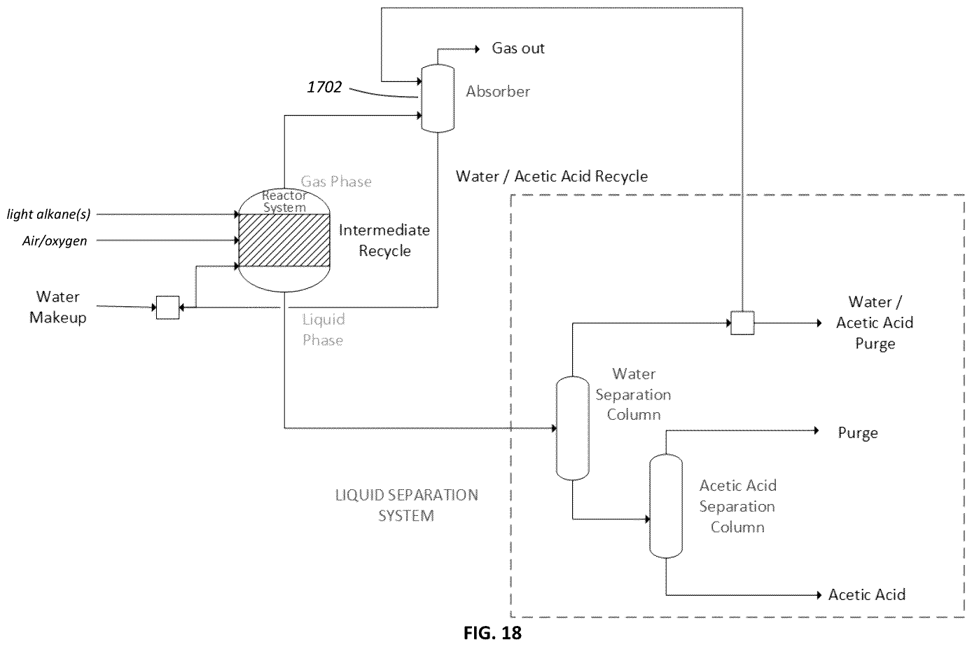

In some embodiments, no entrainer is used to separate acetic acid and water as in the embodiments of FIGS. 9 and 10, and the water recycle can include a small amount of acetic acid. An example of such a process flowsheet is shown in FIG. 11 in which acetic acid is recovered without an entrainer. Feed streams, reactor, and lights separation portion are the same as or similar to those described above with respect to FIG. 18.

Referring to FIG. 11, stream 1002 goes to the water removal column. Typical operating pressures in the water column are between about 0.5 atm (0.5 bar) to about 12 atm, (12 bar) or about 7 atm (7 bar). A water and acetic acid stream having mostly water is obtained in the distillate. A fraction of this stream is purged in splitter 1004 to avoid build-up of water and organic impurities in the process of this stream whereas the rest is recycled to the reactor. The pure acetic acid product can be 99 wt % or greater in the bottoms product of the water removal column. Because the reaction produces water, a fraction of the pure water product is purged from the process, while the rest is recycled to the reactor system. Note that the purge in this case does not contain a significant amount of acetic acid. Consequently, the overall acetic acid recovery is higher than that for the separation sequences without an entrainer, but at a cost of additional columns.

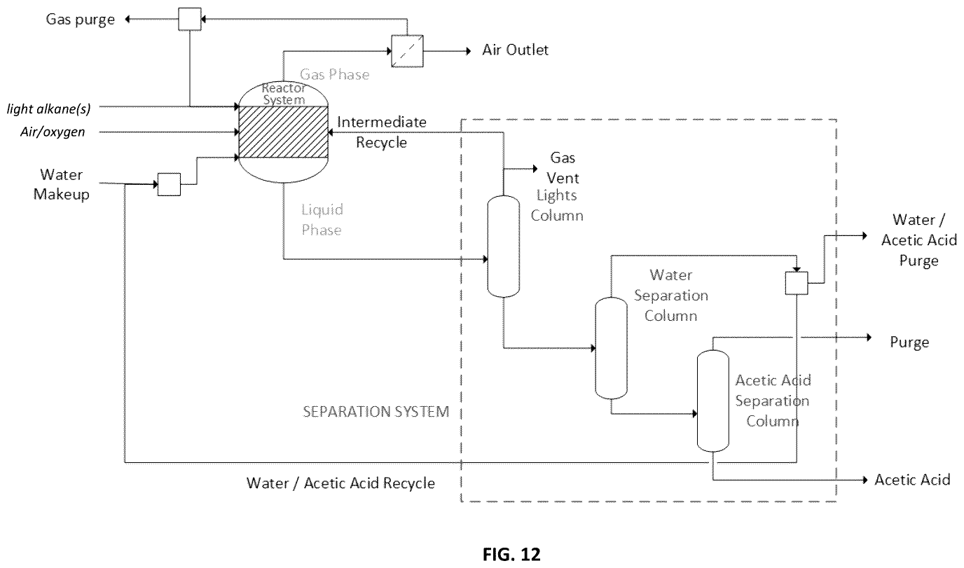

A schematic of the process flowsheet is shown in FIG. 12 in which high purity acetic acid product can be recovered without the use of an entrainer, along with a small amount of by-product impurity. The feed streams, reactor, and lights separation are the same as or similar to those described with respect to FIGS. 10 and 11.

Referring to FIG. 12, in some embodiments, no entrainer is used to separate the acetic acid and water as in the first example, and the water recycle can include a small amount of acetic acid. Typical operating pressures in the water column can be between about 0.5 atm (0.5 bar) to about 12 atm, (12 bar) or about 7 atm (7 bar). The acetic acid column 1104 can be operated at approximately atmospheric pressure. After separating water (with the rest being predominantly acetic acid with small amounts of other impurities and/or reaction intermediates) in the distillate of the water column (as seen in FIG. 12), impurities can be purged in the distillate of the acetic acid column 1104 (along with a small loss of acetic acid). Pure acetic acid product can be obtained at the bottom of the acetic acid column 1104 at a purity of about 99.9 wt % or greater.

The order of the water and acetic acid separation columns shown in FIG. 12 can also be reversed with the acetic acid being recovered as a bottom product in the first water column 1102, the water-acetic acid recycle stream can be the distillate of the second acetic acid column 1104, and the purge can be taken as the bottom product of the second acetic acid column 1104. In this case, the operating pressures of both the columns can be between about 0.5 atm (0.5 bar) to about 12 atm, (12 bar) or about 7 atm (7 bar). In addition, both options can be combined into a single column with the water acetic acid stream as a distillate, acetic acid as a bottom product, and the purge taken as a side-draw. A combined column's operating pressure can be between about 0.5 atm (0.5 bar) to about 12 atm, (12 bar) or about 7 atm (7 bar).

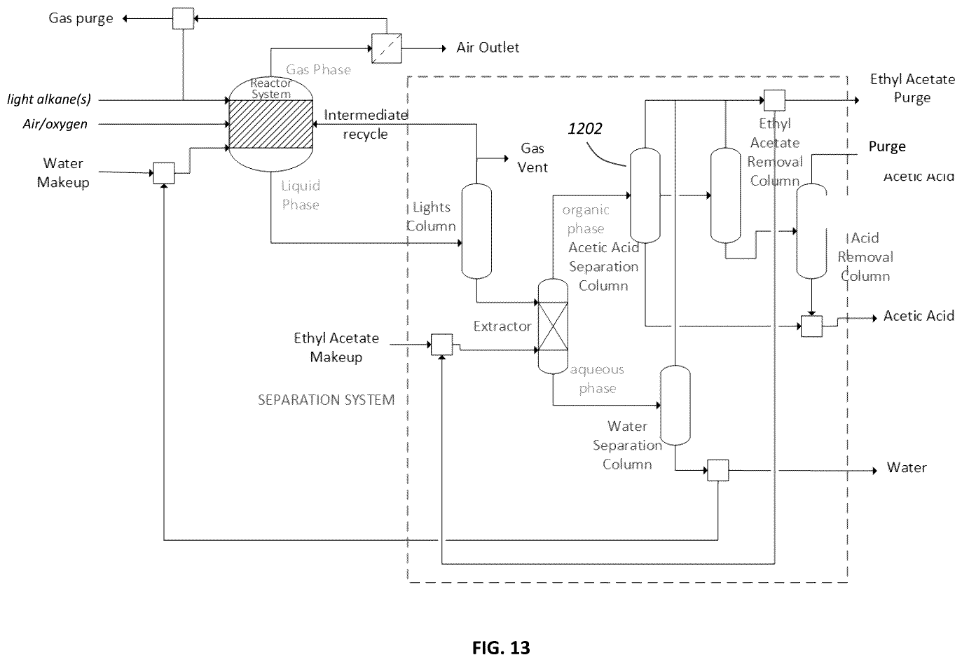

Another schematic process flowsheet example is shown in FIG. 13 in which high purity acetic acid product can be recovered with the use of an entrainer (e.g., ethyl acetate, etc.), along with a small amount of by-product impurity. The feed streams, reactor, and lights separation are the same as described with respect to FIGS. 10 and 11. In this example, when the entrainer is used to separate water and acetic acid, additional columns can be used to obtain high purity water (e.g., 99.9 wt % or greater water) and high purity acetic acid (e.g., 99.9 wt % or greater acetic acid) products. Acetic acid and water are removed in a side-draw from the acetic acid separation column 1202, which are further treated to remove an acetic acid via a purge stream (with 1 wt % loss of acetic acid) and pure acetic acid product.

For separation sequences with or without entrainer, the lights column used for degassing can also be replaced, depending on the separation sequence, with various configurations. For example, a partial condenser followed by a flash vessel/reflux accumulator can be used instead of the lights column. Any dissolved gases can be removed in the vapor phase, while light reaction intermediates such as ethanol and acetaldehyde can be recovered in the liquid phase and recycled back to the reactor. Other options can include a three-phase decanter, a flash vessel, and/or a vent on the extraction column.

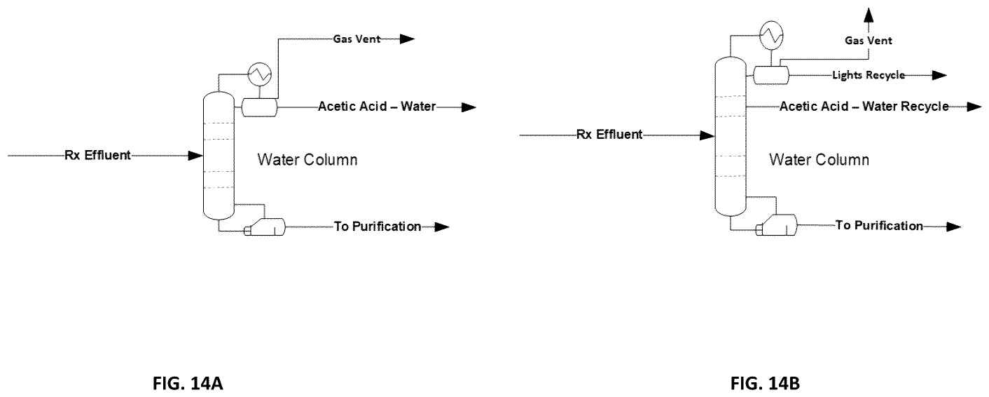

FIGS. 14A and 14B illustrate alternate column configurations showing the gas vent addition to the water column. In FIG. 14A, the light reaction intermediates can be recovered in the acetic acid-water distillate product, while in FIG. 14B they can be recovered separately as the distillate (lights recycle), while the acetic-acid water recycle is recovered as a side stream from the column.

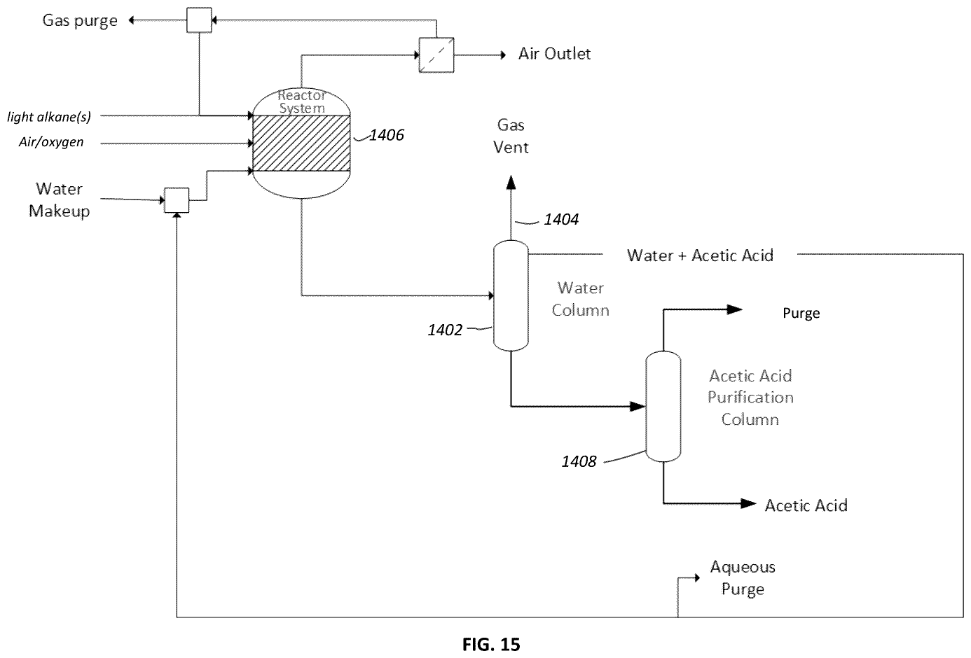

FIG. 15 illustrates an extension of FIG. 14A to the entire separation sequence. The general sequence can include any of those shown in FIGS. 11-12, and the corresponding components can be the same or similar to those described above with respect to FIGS. 11 and 12. FIG. 15 illustrates the separation of acetic acid product from water with no entrainer, without a degassing column, and without a separate lights recycle. The light reaction intermediates can be recovered in the water-acetic acid distillate and recycled to the reactor after an aqueous purge to remove the water generated in the reactor.

More specifically, FIG. 15 illustrates a process flowsheet for the separation of acetic acid and water (and containing by-product impurity). The feed streams and reactor configurations can be the same or similar to the arrangement as described with respect to FIG. 12. However, the degassing or lights column can be removed and a gas vent stream 1404 can be introduced on the water column 1402. In this case, the light reaction intermediates can be recycled to the reactor 1406 within the water-acetic acid recycle. Typical operating pressures within the water column 1402 can be between about 0.5 atm (0.5 bar) to about 12 atm, (12 bar) or about 7 atm (7 bar). The acetic acid column 1408 can be operated at or near atmospheric pressure.

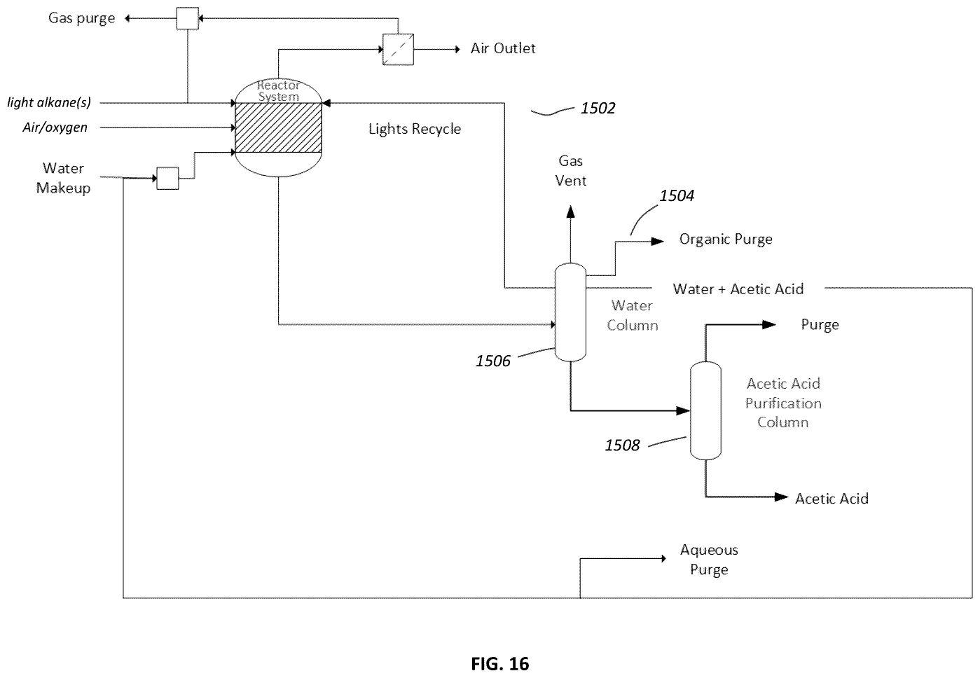

FIG. 16 illustrates an extension of FIG. 14B to the entire separation sequence. The general sequence can include any of those shown in FIGS. 11-12, and the corresponding components can be the same or similar to those described above with respect to FIGS. 11 and 12. FIG. 16 shows the separation of acetic acid product from water with no entrainer, without a degassing column but with a lights recycle (comprising primarily light reaction intermediates) to the reactor and an optional organic purge to remove any light reaction by-products and impurities from the process. FIG. 16 shows a configuration that is similar to that shown in FIG. 15 (where like components can be the same or similar) but with a separate lights recycle in stream 1502 and an optional organic purge in stream 1504. Typical operating pressures within the water column 1506 can be between about 0.5 atm (0.5 bar) to about 12 atm, (12 bar) or about 7 atm (7 bar). The acetic acid column 1508 can be operated at about atmospheric pressure.

When no entrainer is used (e.g., in any of the schemes in FIGS. 11-16), water can be recovered as the distillate of the water column 1506 and recycled back to the reactor after a purge. While it is theoretically possible to achieve a pure (>99 wt %) water distillate, such recovery may not be practical or economical, and the distillate can contain some acetic acid, and possibly some amount of impurities as well. Consequently, the aqueous purge can result in a small acetic acid loss, and may be further separated (e.g., using azeotropic distillation) in order to increase the overall acetic acid recovery.

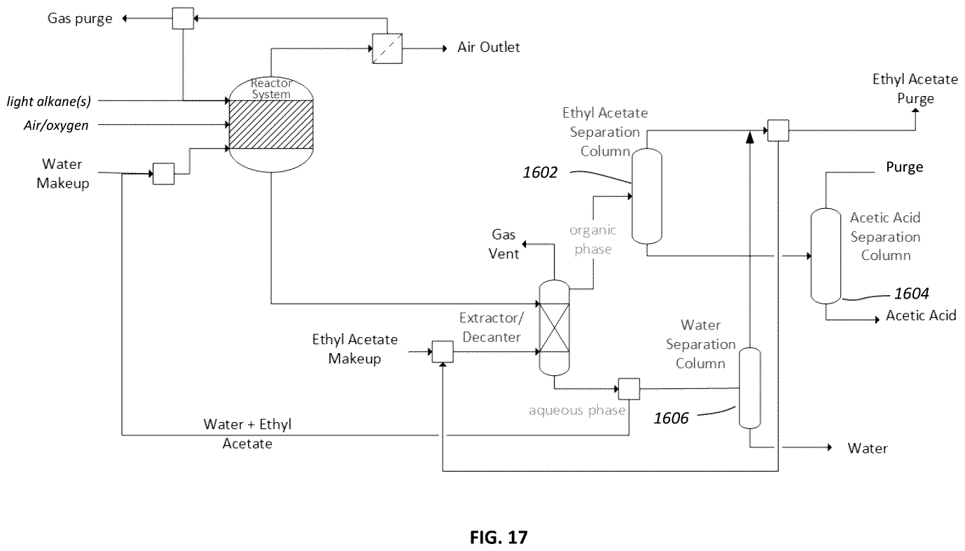

A schematic of a process flowsheet is shown in FIG. 17. The feed streams and reactor can be the same or similar to those shown in the same as in FIG. 13, and like components will not be re-described in the interest of brevity. The main difference in FIG. 13 is that the degassing or lights column can be removed and a gas vent can be introduced on the extractor. In this embodiment, the bulk of the light reaction intermediates can be recycled to the reactor within the water recycle, while a small fraction may be lost with the ethyl acetate purge. Unlike the configuration in FIG. 13, the acetic acid can be removed as a bottom product from the ethyl acetate removal column 1602, whereas ethyl acetate and water can be removed as a distillate of the same. The impurities can be purged from the distillate of the next column 1604 (with a small loss of acetic acid) and pure or nearly pure acetic acid product can be obtained as a bottom product. To reduce the energy consumption of the water separation column 1606, some of the water (saturated with ethyl acetate) can be recycled to the reactor before this distillation.

In any of the embodiments described herein, products or by-products having a heavier molecular mass than acetic acid may be produced when hydrocarbons heavier than ethane are present in the feed to the reaction. Any by-products heavier than acetic acid can pass through the separation system and be present in the acetic acid product stream. Any suitable downstream separation can be used to produce high purity acetic acid by removing heavier by-products from the acetic acid such as distillation, extraction, and the like.

Another schematic of the process is shown in FIG. 18. This flow sheet is similar to the flowsheet shown in FIG. 11. Feed streams, reactor and separation columns are the same as FIG. 11 but with an absorber 1702 on the gas exiting the reactor and a gas exit stream instead of a gas recycle.

In some aspect, any of the separation schemes described herein can be used with any suitable reactor arrangements.

### EXAMPLES

The subject matter having been generally described, the following examples are given as particular aspects of the disclosure and are included to demonstrate the practice and advantages thereof, as well as preferred aspects and features of the inventions. It is understood that the examples are given by way of illustration and are not intended to limit the specification of the claims to follow in any manner.

### Comparison Example 1

An amount of 6.5 gram (g) of Mo_{0.62}V_{0.32}Nb_{0.06}Oₓ/TiO₂ catalyst is made as follows:

Solution A: 0.46 g of NH₄NO₃ (ammonium metavanadate) is added to a 100-milliliter (mL) size of glass flask. Next 10 mL of DI-H₂O is added into the flask. Stirring is started at ambient temperature until the ammonium metavanadate is completely dissolved. An amount of 0.90 g of oxalic acid powder is gradually added to the solution. An amount of 1.35 g of (NH₄)₆Mo₇O₂₄·24H₂O solid (ammonium heptamolybdate tetrahydrate) is added to the above solution while stirring at ambient temperature.

After mixing for 10 minutes, 5.00 g of titania powder (BET area: 54 meter squared per gram (m²/g)) is added to the above solution.

Solution B: 0.31 g C₄H₄NNbO₉·xH₂O (ammonium niobate (V) oxalate hydrate) is added in 5.0 mL of DI-H₂O.

Solution B is added dropwise to the suspension with solution A and titania. After the total amount of solution B is added, stirring is continued for 5 minutes.

A rotavapor is used until the water is evaporated. The resulting paste is then dried in an oven at 120° C. for 16 hours. Finally, the dried sample is calcined with a ramping calcination temperature from room temperature to 400° C. with 3° C./min ramping rate, and then is maintained at 400° C. for 4 hours.

An amount of 0.3 wt % Pd/SiO₂ is prepared as follows:

An amount of 0.086 g of 10 wt % Pd(NH₃)₄(NO₃)₂(tetraamminepalladium (II) nitrate) is dissolved into 2 mL of DI-H₂O in a beaker to make a yellow solution.

An amount of 1.00 g SiO₂ in powder form is added to the above solution. The mixture in the water bath, which is preheated to 80° C., is dried. This dry powder is transferred into an oven, which is preheated to 120° C., and is maintained at this temperature for 16 hours. The dried sample is then transferred to the muffle furnace for calcination by increasing the calcination temperature from room temperature to 500° C. with 3° C. per minute ramping rate and is maintained at 500° C. for 4 hours.

Physical mixtures of 0.3 wt % Pd/SiO₂ and Mo_{0.62}V_{0.32}Nb_{0.06}O/TiO₂ catalysts are prepared by grinding mixed powders with an agate mortar and pestle and then pressing into wafers and sieving to the desired size. The final mixture contains 0.01 wt % Pd.

### Example 2

An amount of 6.5 g of Mo_{0.62}V_{0.32}Nb_{0.06}O/TiO₂ catalyst is made as follows:

Solution A: 0.46 g of NH₄NO₃ (ammonium metavanadate) is added to a 100-mL size of glass flask. An amount of 10 mL of DI-H₂O is then added into the flask. Stirring is started at ambient temperature until the ammonium metavanadate is completely dissolved. An amount of 0.91 g of oxalic acid power is gradually added to the solution. An amount of 1.35 g of (NH₄)₆Mo₇O₂₄·24H₂O solid (ammonium heptamolybdate tetrahydrate) is added to the above solution while stirring at ambient temperature.

Solution B: an amount of 0.31 g C₄H₄NNbO₉ XH₂O (ammonium niobate (V) oxalate hydrate) is added in 5.0 mL of DI-H₂O.

An amount of solution B is added dropwise to solution A. After the total amount of solution B is added, stirring is maintained for 5 minutes.

An amount of 5.01 g of titania powder (BET area: 54 m²/g) is added to the above solution and keep stirring for additional 10 minutes.

A rotavapor evaporates water until consistent weight is obtained (within 0.1 g of previous weight). The resulting paste is dried in an oven at 120° C. for 16 hours. Finally, the dried sample is calcined with a ramping calcination temperature from room temperature to 400° C. with 3° C./min ramping rate, and then is maintained at 400° C. for 4 hours.

An amount of 0.3 wt % Pd/SiO₂ is prepared by the following:

An amount of 0.08416 g of 10 wt % Pd(NH₃)₄(NO₃)₂(tetraamminepalladium (II) nitrate) aqueous solution is diluted into 2 g of DI-H₂O in a beaker to make a yellow solution.

An amount of 1 g SiO₂ in powder form is added to the above solution. The mixture in the water bath, which is preheated to 80° C., is dried. This dry powder is transferred into the oven, which is preheated to 120° C., and is maintained at this temperature for 16 hours. The dried sample is transferred to the muffle furnace for calcination by increasing the calcination temperature from room temperature to 500° C. with 3° C. per minute ramping rate and is maintained at 500° C. for 4 hours.

Physical mixtures of 0.067 g of 0.3 wt % Pd/SiO₂ and 2.01 g of Mo_{0.62}V_{0.32}Nb_{0.06}O—/TiO₂ catalysts are prepared by grinding mixed powders with an agate mortar and pestle and then pressing into wafers and sieving to the desired size. The final mixture contains 0.01 wt % Pd.

### Example 3

The same preparation protocol is used in this example as in Example 2 with only one exception: an amount of 2.51 g of titania power is used.

### Example 4

The same preparation protocol is used in this example as in Example 2 with only one exception: an amount of 7.51 g of titania power is used.

### Example 5

An amount of 6.5 g of Mo_{0.62}V_{0.32}Nb_{0.06}O/TiO₂ catalyst is made as follows:

Solution A: 0.46 g of NH₄NO₃ (ammonium metavanadate) is added to a 100-mL size of glass flask. An amount of 10 mL of DI-H₂O is added into the flask. Stirring at ambient temperature is started until the ammonium metavanadate is completely dissolved. An amount of 0.91 g of oxalic acid power is added to the solution. An amount of 1.35 g of (NH₄)₆Mo₇O₂₄·24H₂O solid (ammonium heptamolybdate tetrahydrate) is added to above solution while stirring at ambient temperature.

Solution B: An amount of 0.31 g C₄H₄NNbO₉ xH₂O (ammonium niobate (V) oxalate hydrate) is added in 5.0 mL of DI-H₂O.

A solution B is added dropwise to solution A. After the total amount of solution B is added, stirring is maintained for 5 minutes.

An amount of 5.01 g of titania powder (BET area: 54 m²/g) is added to the above solution and stirring is maintained for additional 10 minutes.

A rotavapor evaporates water until consistent weight is obtained (within 0.1 g of previous weight). The resulting paste is then dried in an oven at 120° C. for 16 hours. Finally, the dried sample is calcined with a ramping calcination temperature from room temperature to 400° C. with 3° C./min ramping rate, and is then maintained at 400° C. for 4 hours.

An amount of 0.018 g of 10 wt % Pd(NH₃)₄(NO₃)₂(tetraamminepalladium (II) nitrate) aqueous solution is then further diluted into 4 g of DI-H₂O in a beaker to make a yellow solution. Stirring is maintained for 5 minutes.

The palladium solution is added to 2.00 g of Mo_{0.62}V_{0.32}Nb_{0.06}O/TiO₂ in powder form. The mixture in the water bath, which is preheated to 80° C., is dried. This dry powder is transferred into the oven, which is preheated to 120° C., and is maintained at this temperature for 16 hours. The dried sample is then transferred to the muffle furnace for calcination by increasing the calcination temperature from room temperature to 400° C. with 3° C. per minute ramping rate and keep at 400° C. for 4 hours. The final catalyst contains 0.03 wt % Pd.

### Example 6

An amount of 6.5 g of Mo_{0.60}V_{0.31}Nb_{0.05}Ce_{0.04}Oₓ/TiO₂ catalyst is made as follows:

Solution A: 0.46 g of NH₄NO₃ (ammonium metavanadate) is added to a 100-mL size of glass flask. An amount of 10 mL of DI-H₂O is then added into the flask. Stirring is started at ambient temperature until the ammonium metavanadate is completely dissolved. An amount of 0.90 g of oxalic acid power is added to the solution. An amount of 1.35 g of (NH₄)₆Mo₇O₂₄·24H₂O solid (ammonium heptamolybdate tetrahydrate) is added to above solution while stirring at ambient temperature. An amount of 0.203 g of Ce(NO₃)₃·6H₂O(Cerium (III) nitrate hexahydrate) is added to the above solution.

Solution B: Add 0.31 g C₄H₄NNbO₉: xH₂O (ammonium niobate (V) oxalate hydrate) in 5.0 mL of DI-H₂O.

An amount of solution B is added dropwise to solution A. After the total amount of solution B is added, stirring is maintained for 5 minutes.

An amount of 5.01 g of titania powder (BET area: 54 m²/g) is added to the above solution and stirring is maintained for additional 10 minutes.

A rotavapor evaporates water until consistent weight is obtained (within 0.1 g of previous weight). The resulting paste is then dried in the oven at 120° C. for 16 hours. Finally, the dried sample is calcined with a ramping calcination temperature from room temperature to 400° C. with 3° C./min ramping rate, and then is maintained at 400° C. for 4 hours.

An amount of 0.3 wt % Pd/SiO₂ is prepared by the following steps:

An amount of 0.08416 g of 10 wt % Pd(NH₃)₄(NO₃)₂(tetraamminepalladium (II) nitrate) aqueous solution is then further diluted into 2 g of DI-H₂O in a beaker to make a yellow solution.

An amount of 1 g SiO₂ in powder form is added to the above solution. The mixture in the water bath, which is preheated to 80° C., is dried. This dry powder is transferred into the oven, which is preheated to 120° C., and is maintained at this temperature for 16 hours. The dried sample is then transferred to the muffle furnace for calcination by increasing a calcination temperature from room temperature to 500° C. with 3° C. per minute ramping rate and keep at 500° C. for 4 hours.

Physical mixtures of 0.067 g of 0.3 wt % Pd/SiO₂ and 2.01 g of Mo_{0.60}V_{0.31}Nb_{0.05}Ce_{0.04}Oₓ/TiO₂ catalysts are prepared by grinding mixed powders with an agate mortar and pestle and then pressing into wafers and sieving to the desired size. The final mixture contains 0.01 wt % Pd.

### Example 7

The catalyst performance test is carried out with a tubular reactor with 12.7 mm outer diameter (OD), 9.0 mm inch internal diameter (ID), and 254 mm length. It is surrounded by a brass block. The block is surrounded by a band heater. Reactor temperature is measured by an internal thermocouple that located in the center of the reactor.

Catalyst loading: glass wool is applied to the bottom of the reactor, followed by adding 4 mm size of glass beads and then adding 1 mm size of glass beads. All the glass beads are treated with 5% nitric acid and rinsed with DI-water until the pH is near 7. An amount of 0.5 g of catalyst MO_{0.62}V_{0.32}Nb_{0.06}O/TiO₂+Pd/SiO₂ from example 2 is well mixed with 3 g of 1 mm glass bead and then is added into the reactor followed by adding 1 mm size of glass beads, 4 mm size of glass beads and glass wool to form the catalyst bed. The center of catalyst bed is ensured to align with the tip of internal thermal couple.

After the reactor is connected to the test unit, the pressure check is implemented with nitrogen. The system is then purged with nitrogen until oxygen free. The reactor temperature is set to 300° C. with the actual temperature control at 300±1° C. The ethane flow is initially established at 8 standard cubic centimeter per minute (sccm) and then air flow at 10 sccm. A HPLC pump is used to introduce 0.01 cubic centimeter per minute (cc/min) of type I water from the reactor inlet. The pressure reactor system is controlled at 232 psi (16.0 bar) through a back pressure regulator.

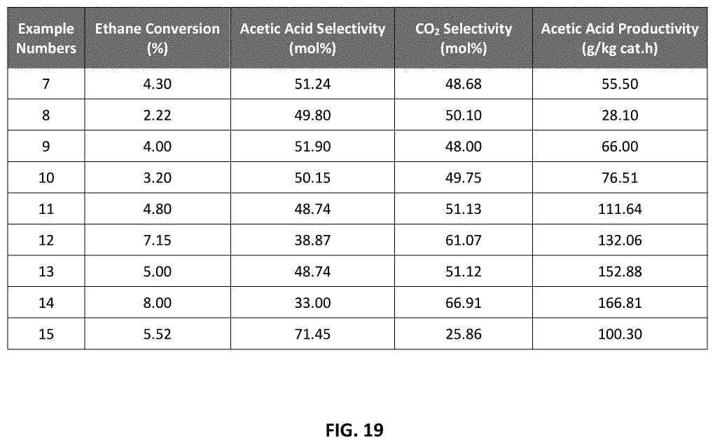

The liquid product is condensed in a condenser and routinely collected for GC analysis. The vent gas is also collected at atmospheric pressure for GC analysis. The calculated test results based on the GC analytical results are listed in FIG. 19.

### Comparison Example 8

The same testing protocol as Example 7 is used in this example with only one exception: the catalyst from Comparison Example 1 is used. The calculated test results based on the GC analytical results are listed in FIG. 19.

### Example 9

The same testing protocol as Example 7 is used in this example with exceptions: the ethane flow is 10 sccm and air flow is 30 sccm. The calculated test results based on the GC analytical results are listed in FIG. 19.

### Example 10

The same testing protocol as Example 7 is used in this example with exceptions: the ethane flow is 15 sccm, air flow is 45 sccm, and the water pumping rate is 0.015 cc/min. The calculated test results based on the GC analytical results are listed in FIG. 19.

### Example 11

The same testing protocol as Example 10 is used in this example with one exception: the reaction temperature is 315° C. The calculated test results based on the GC analytical results are listed in FIG. 19.

### Example 12

The same testing protocol as Example 10 is used in this example with one exception: the reaction temperature is 330° C. The calculated test results based on the GC analytical results are listed in FIG. 19.

### Example 13

The same testing protocol as Example 12 is used in this example with exceptions: the ethane flow is 20 sccm, air flow is 60 sccm, and the water pumping rate is 0.02 cc/min. The calculated test results based on the GC analytical results are listed in FIG. 19.

### Example 14

The same testing protocol as Example 13 is used in this example with one exception: the reaction temperature is 345° C. The calculated test results based on the GC analytical results are listed in FIG. 19.

### Example 15

The same testing protocol as Example 7 is used in this example with exceptions: the catalyst MO_{0.60}V_{0.31}Nb_{0.05}Ce_{0.04}Oₓ/TiO₂+Pd/SiO₂ from Example 6 is used, and water pumping rate is 0.005 cc/min. The calculated test results based on the GC analytical results are listed in FIG. 19.

### Example 16

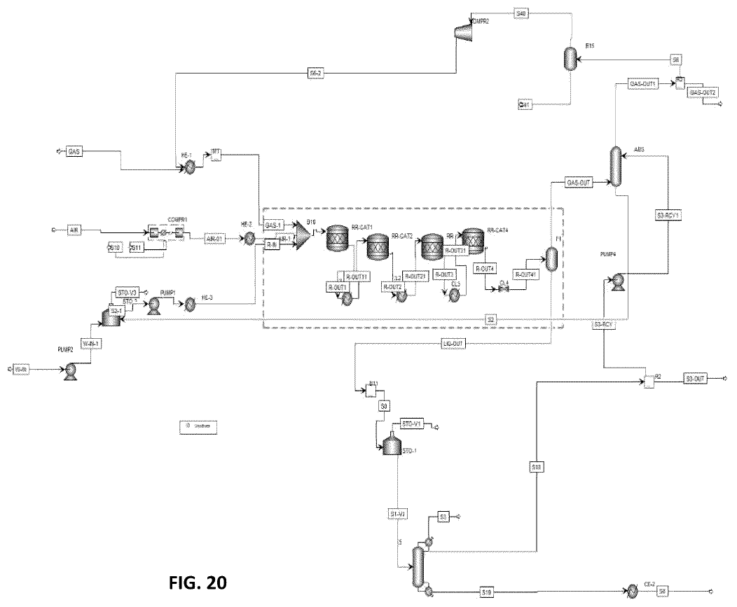

As an example of the process, the process flowsheet shown in FIG. 20 shows a simulated process flow that was simulated using Aspen Plus process design software, version 12.1. The simulated process is similar to the schematic for the process shown in FIG. 4.

Feed streams are indicated as follows: GAS (with a composition of 99.5% v/v ethane and 0.5% v/v propane), and AIR (79 vol. % N₂, 21 vol. % O₂),

The AIR stream is compressed in a three-stage compressor COMPR1 to 15 atm (15 bar), and subsequently heated to 250° C. The GAS stream is combined with the recycle stream S6-2, and the combined stream is preheated to 250° C. The water recycle stream S2 is pressurized to 15 atm (15 bar) using PUMP1, and subsequently vaporized and superheated to 250° C. All three streams are then introduced to the Reactor System.

The Reactor System shown in FIG. 20 is a very simplified schematic of the adiabatic reactor cascade described in paragraphs [0054]-[0058], designed to illustrate that (a) the Reactor SYSTEM comprises of series of four adiabatic reactors; (b) the temperature of the feed to each reactor is 250° C.; (c) the temperature of gas stream existing each reactor is 350° C.; and (b) the gas stream leaving each reactor is cooled before it is fed to the next reactor. FIG. 20 is simplified and does not show that AIR being split and fed to each reactor separately and that the maximum temperature in each reactor being controlled by changing the AIR flow to each reactor. The gas stream exiting reactor RR-CAT4 is cooled to 60° C. and sent to flash drum F1, where the gas and liquid phases are separated.

The vapor stream from flash drum F1 is sent to the absorber ABS, where is it contacted with the recycled water stream S3-RCY1 from the water acetic acid separation column B5 to recover the bulk of the acetic acid present in the vapor stream (of the 600 kilogram per hour (kg/hr) of acetic acid present in the feed to the absorber, ˜570 kg/hr is recovered). The bottoms stream (S2) from the absorber is recycled to the reactor system, and comprises 63 wt % water and 37 wt % acetic acid with a flow of 1556 kg/h. Approximately 77% of the vapor stream leaving the absorber ABS is compressed and recycled to the reactor system, while the rest is purged from the process. Purge GAS-OUT2 and the recycle stream S6-2 comprise 10% ethane, 82% nitrogen, 2% oxygen (O₂) and 2% water

Stream S1 is the feed to the water removal column, wherein 95 wt % water (containing ˜5 wt % acetic acid) is recovered as the distillate S18 at 40° C. and 99.96 wt % pure Acetic acid is recovered as the bottoms product stream S19 at 195° C., which is subsequently cooled to 40° C. in cooler CE-2. Approximately 50% of distillate S18 is sent to the absorber ABS and the rest is purged out in stream S3-OUT. Column B5 is operated at 7 atm (7 bar).

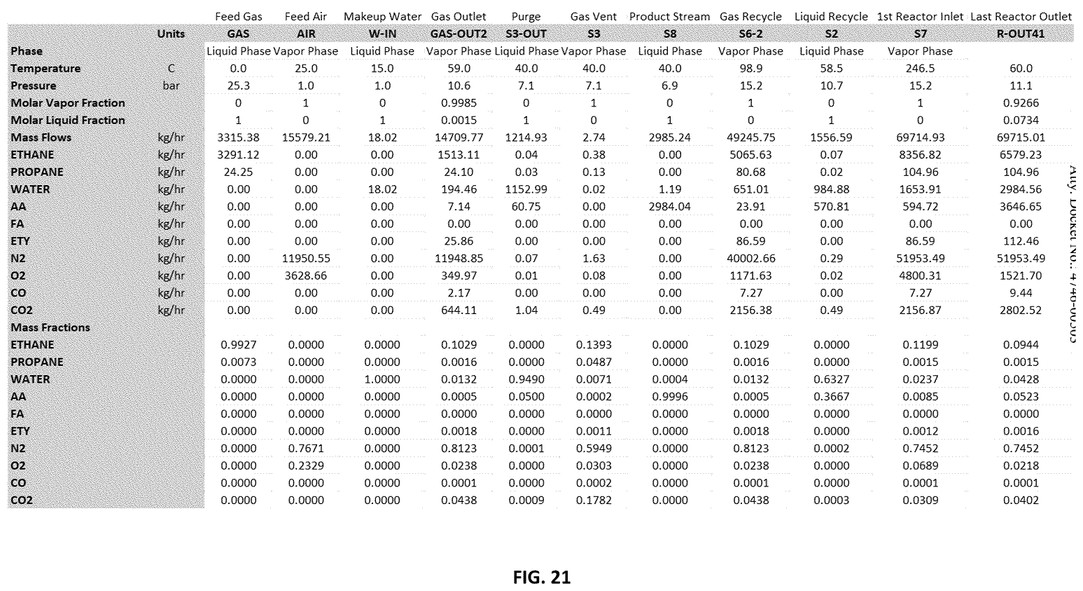

The overall mass balance with the inlet and outlet streams, as well as select process streams, is shown in FIG. 21. The overall mass balance with the inlet and outlet streams is shown in FIG. 21 for the flowsheet described above and in FIG. 20.

### Example 17

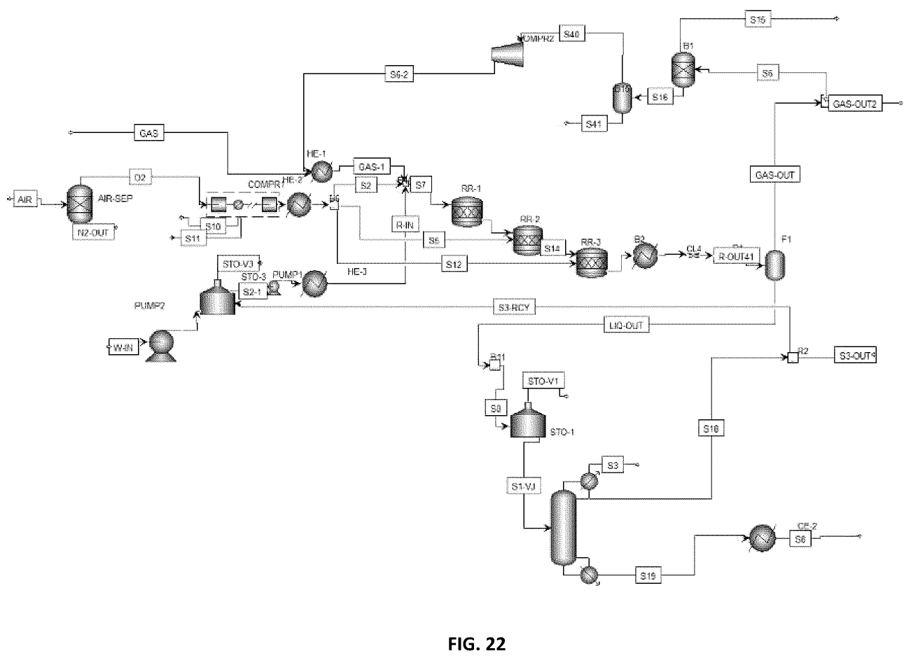

As an example of an isothermal process, the process flowsheet shown in FIG. 22 shows a simulated process flow that was simulated using Aspen Plus process design software, version 12.1. The simulated process is similar to the schematic for the process shown in FIG. 8.

Feed streams are indicated as follows: GAS (with a composition of 99.5% v/v ethane and 0.5% v/v propane), and AIR (79 vol. % N2, 21 vol. % O2). An air separator is used to separate nitrogen from air such that pure oxygen is sent to the process at 99% concentration.

The oxygen stream is compressed in a three-stage compressor COMPR1 to 30 atm (30 bar), and subsequently heated to 275° C. with the recycled gas. The GAS stream is combined with the recycle stream S6-2, and the combined stream is preheated to 275° C. The water recycle stream S2 is pressurized to 30 atm (30 bar) using PUMP1, and subsequently vaporized and superheated to 275° C. The net gas and water streams are introduced to the first reactor in the cascade, whereas the pressurized oxygen stream is distributed into each of the three reactors in the cascade in streams S2, S5, S12.

The Reactor System shown in FIG. 22 is a schematic of an isothermal reactor cascade described with respect to FIG. 8 designed to illustrate that (a) the reactor system comprises of series of 3 isothermal reactors; (b) the temperature of each reactor in the cascade is 275° C., and there are no intercoolers; (c) the air flow, however, is distributed to each reactor such that almost all the oxygen is consumed in each reactor. The gas stream exiting the last reactor in the cascade RR-3 is cooled to 60° C. and sent to flash drum F1, where the gas and liquid phases are separated.

The vapor stream from flash drum F1, after a 2% purge is sent to a carbon dioxide scrubber that removes carbon dioxide almost completely and recycles unreacted ethane, a small fraction of the non-condensables that were present in the feed. This stream is then compressed in COMPR2 to 30 atm (30 bar). Purge GAS-OUT2 comprises 77% ethane, 15% nitrogen, and 7% carbon dioxide. The recycle stream S6-2 contains 83% ethane and 17% nitrogen.

The liquid stream, LIQ-OUT, from the flash drum F1 sent to the water acetic acid separation column containing 52 wt % water and 47 wt % acetic acid, in which 94 wt % water (containing ˜5 wt % acetic acid) is recovered as the distillate S18 at 40° C. and 99.96 wt % pure Acetic acid is recovered as the bottoms product stream S19 at 195° C., which is subsequently cooled to 40° C. in cooler CE-2 in exit stream S8. Approximately 44% of distillate S18 is purged out in stream S3-OUT and the rest is recycled in S3-RCY to the reaction system as it contains 95 wt % water and 5% acetic acid. Column B5 is operated at 7 atm (7 bar).

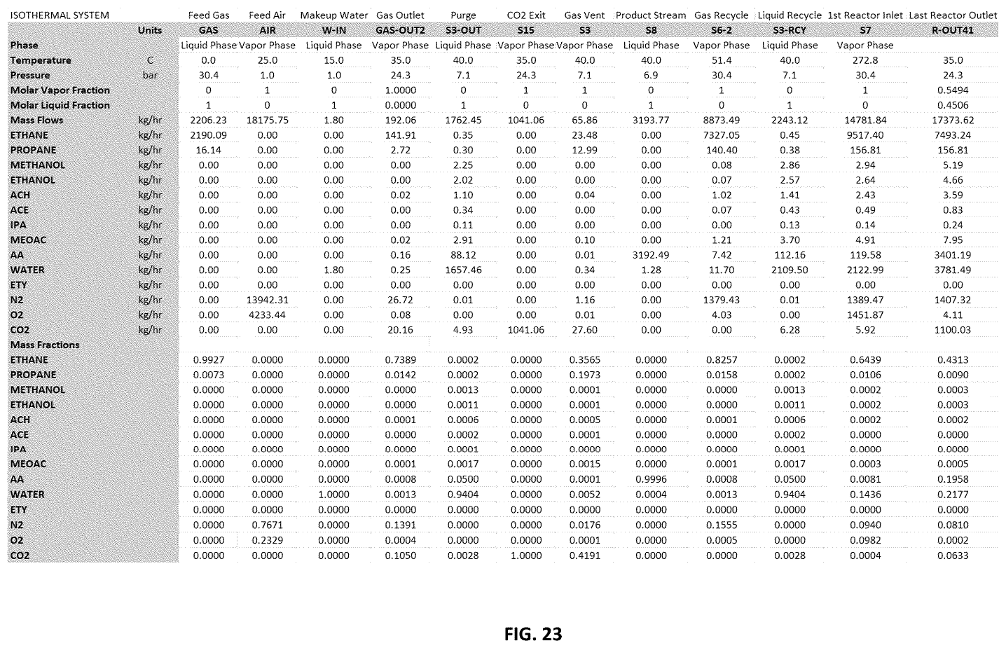

The overall mass balance with the inlet and outlet streams, as well as select process streams, is shown in FIG. 23.

Having described various processes and systems, certain aspects can include, but are not limited to:

In a first aspect, a process to make oxygenates from light alkanes comprises a reactor system and a product separation system, wherein the reactor system comprises one or two catalysts, wherein the catalysts contain molybdenum, vanadium, niobium, cerium, titanium, zirconium, precious metals, and/or oxides thereof.

A second aspect can include the process of the first aspect, wherein there is only one catalyst used for the reactors, and wherein the catalyst comprises the oxides of molybdenum, vanadium, niobium, cerium, titanium, zirconium, and precious metals.

A third aspect can include the process of the first aspect, wherein there is only one catalyst used for the reactors. The catalyst contains the oxides of molybdenum, vanadium, niobium, cerium, titanium, and precious metals.

A fourth aspect can include the process of the first aspect, wherein there is only one catalyst used for the reactors. The catalyst contains the oxides of molybdenum, vanadium, niobium, titanium, and precious metals.

A fifth aspect can include the process of the first aspect, wherein there are two catalysts used for the reactors. The first catalyst contains the oxides of molybdenum, vanadium, niobium, cerium, titanium, zirconium, and the second catalyst contains supported precious metals.

A sixth aspect can include the process of the first aspect, wherein there are two catalysts used for the reactors. The first catalyst contains the oxides of molybdenum, vanadium, niobium, cerium, titanium, and the second catalyst contains supported precious metals.

A seventh aspect can include the process of the first aspect, wherein there are two catalysts used for the reactors. The first catalyst contains the oxides of molybdenum, vanadium, niobium, titanium, and the second catalyst contains supported precious metals.

An eighth aspect can include the process of the first aspect, wherein at least one catalyst comprises of the crystallite of molybdenum, niobium, vanadium oxides.

A ninth aspect can include the process of the first aspect, wherein the precious metals comprise any one of ruthenium (Ru), rhodium (Rh), palladium (Pd), osmium (Os), iridium (Ir), platinum (Pt), gold (Au), and any combination thereof.

A tenth aspect can include the process of any one of the fifth to seventh aspects, wherein the support for the precious metals includes titania, alumina, silica, and any combination thereof.

An eleventh aspect can include the process of any one of the fifth to seventh aspects, wherein the catalysts can be uniformly mixed prior to the catalyst loading; in some embodiments, the different catalyst can be loaded separately as stacked bed within one reactor, or separately loaded in different reactors.

A twelfth aspect can include the process of any one of the first to eleventh aspects, wherein the reactor type includes any one of fixed bed reactor, continues stirred tank reactor (CSTR), fluidized bed reactor, moving bed reactor, continuous catalyst regeneration (CCR) reactor, or the combination thereof.

A thirteenth aspect can include the process of any one of the first to twelfth aspects, wherein the light alkanes include methane, ethane, propane, isobutane.

A fourteenth aspect can include the process of any one of the first to thirteenth aspects, wherein the light alkanes are selectively converted to the oxygenates in the presence of oxygen containing gas and water.

A fifteenth aspect can include the process of any one of the first to fourteenth aspects, wherein the operation temperature of reactor system is in the range of 220° C. to 600° C., especially in the range of 240° C. to 500° C., especially in the range of 260° C. to 450° C.

A sixteenth aspect can include the process of any one of the first to fifteenth aspects, wherein the operation pressure of reactor system is in the range of 50 psi to 3000 psi, especially in the range of 100 psi to 2500 psi, especially in the range of 200 psi to 2,000 psi.

A seventeenth aspect can include the process of any one of the first to sixteenth aspects, wherein the gas hourly space velocity (GHSV) of reactor system is in the range of 500 h⁻¹ to 20,000 h⁻¹, especially in the range of 1,000 h⁻¹ to 15,000 h⁻¹, especially in the range of 2,000 h⁻¹ to 10,000 h⁻¹.

An eighteenth aspect can include the process of any one of the first to seventeenth aspects, wherein the light alkane gas in the gas feed is greater than 5%, alternatively greater than 15%, alternatively greater than 25%, or alternatively greater than 35%.

A nineteenth aspect can include the process of the fourteenth aspect, wherein the molar ratio of water addition to ethane is in the range of 1/50 to 10/1, especially in the rage of 1/25 to 5/1, especially in the range 1/15 to 3/1.

A twentieth aspect can include the process of any one of the first to nineteenth aspects, wherein the ethane conversion in a single path should be higher than 3%, especially higher than 5%, especially higher than 7.5%, especially higher than 10%.

A twenty first aspect can include the process of any one of the first to twentieth aspects, wherein the acetic acid selectivity should be higher than 30 mol %, especially should be higher than 45 mol %, especially should be higher than 60 mol %.

A twenty second aspect can include the process of any one of the first to twenty first aspects, wherein the acetic acid productivity should be higher than 50 g/kg cat.h, especially should be higher than 75 g/kg cat.h, especially should be higher than 100 g/kg cat.h especially should be higher than 150 g/kg cat.h after 100 hours of time on stream.

A twenty third aspect can include a catalyst, comprising an oxide of (Mo_{0.6} Nb_{0.22}V_{0.18})₅O₁₄.

A twenty fourth aspect can include the catalyst of the twenty third aspect, further comprising a support comprising one or more oxides of cerium, titanium, and zirconium.

A twenty fifth aspect can include the catalyst of the twenty third or twenty fourth aspect, wherein the catalyst further comprises at least one precious metal.

A twenty sixth aspect can include the catalyst of the twenty fifth aspect, wherein the at least precious metal comprises ruthenium, rhodium, palladium, osmium, iridium, platinum, gold, or any combination thereof.

A twenty seventh aspect can include the catalyst of the twenty third aspect, wherein the support comprises one or more oxides of titanium, cerium, zirconium, silicon, aluminum, or any combination thereof.

A twenty eighth aspect can include the catalyst of any one of the twenty third to twenty seventh aspects, wherein the support comprises one or more of a titania, a ceria, a zirconia, a silica, an alumina, or any combination thereof.

A twenty ninth aspect can include the catalyst of any one of the twenty third to twenty eighth aspects, wherein the support comprises one or more of a titania, a ceria, a zirconia, or any combination thereof.

A thirtieth aspect can include the catalyst of any one of the twenty third to twenty ninth aspects, wherein the oxide comprises a crystallite with a crystallite size no more than about 80 angstrom.

A thirty first aspect can include the catalyst of any one of the twenty third to thirtieth aspects, wherein the catalyst comprises at least about 10 wt % of the oxide of (Mo_{0.6} Nb_{0.22}V_{0.18})₅O₁₄, no more than about 1 wt % of the at least one precious metal, and at least about 70 wt % of the support based on the total weight of the catalyst.

A thirty second aspect can include the catalyst of the twenty third aspect, further comprising a first catalyst and a second catalyst, wherein the first catalyst comprises the oxide of (Mo_{0.6}Nb_{0.22}V_{0.18})₅O₁₄ comprised with the support and the second catalyst comprises at least one precious metal on another support comprising a titania, silica, alumina, or any combination thereof.

A thirty third aspect can include the catalyst of the thirty second aspect, wherein the weight ratio of the first catalyst to the second catalyst is 30:1.

In a thirty fourth aspect, a method for converting one or more hydrocarbons comprises: feeding a fluid comprising one or more light alkanes to a reactor system, wherein the reactor system comprises a reactor containing a catalyst, comprising the oxide of (Mo_{0.6} Nb_{0.22}V_{0.18})₅O₁₄ comprised with a support.

A thirty fifth aspect can include the method of the thirty fourth aspect, wherein the reactor comprises a shell and tube vessel containing a catalyst bed disposed on a shell side or on a tube side.

A thirty sixth aspect can include the method of the thirty fourth aspect, wherein the reactor system comprises at least two reactors in series with a cooler after a first reactor to cool an effluent of the first reactor before entering a second reactor.

A thirty seventh aspect can include the method of the thirty sixth aspect, wherein the at least two reactors comprises at least four reactors with a cooler after each of the reactors to cool an effluent of each reactor.

A thirty eighth aspect can include the method of any one of the thirty third to thirty seventh aspects, further comprising: providing a gas separation system and a liquid separation system downstream of the reactor system; producing a water stream from a liquid phase in the liquid separation system; and recycling at least a portion of the water stream to the reactor system, wherein the water stream comprises acetic acid.

A thirty ninth aspect can include the method of the thirty eighth aspect, wherein the water stream comprises acetic acid, wherein producing the water stream comprises: distilling the water stream in a distillation column without the use of an entrainer.

A fortieth aspect can include the method of the thirty eighth aspect, further comprising: distilling the liquid phase using azeotropic distillation to remove at least a portion of the liquid phase from the acetic acid and produce an acetic acid product and a liquid stream; providing an entrainer within a distillation column during distilling; providing the liquid stream after distilling to a decanter for recycling an organic phase to the distillation column and an aqueous phase to another distillation column for separating a water stream; and separating the acetic acid product during the distillation.

A forty first aspect can include the method of the fortieth aspect, wherein the entrainer comprises ethyl acetate, propyl acetate, butyl acetate, or any combination thereof.

A forty second aspect can include the method of the fortieth or forty first aspect, further comprising recycling the water stream comprising acetic acid to the reactor system.

A forty third aspect can include the method of the thirty eighth aspect, further comprising: removing a gas phase stream from the reactor in the gas separation system; separating the gas phase stream to produce a lights recycle stream and a gas stream; and recycling at least a portion of one of: the gas phase stream, the lights recycle stream, or the gas stream to the reactor.

A forty fourth aspect can include the method of the forty third aspect, wherein the gas phase stream comprises methane, ethane, propane, butane(s), ethylene, or combinations thereof.

A forty fifth aspect can include the method of the forty third or forty fourth aspect, further comprising passing the gas phase stream through at least one of a membrane assembly and an absorber.

In a forty sixth aspect, a method for converting one or more hydrocarbons comprises: feeding a fluid comprising one or more light alkanes to a reactor system; wherein the reactor system comprises a reactor containing at least one catalyst, the at least one catalyst comprises one or more oxides of molybdenum, vanadium, niobium, cerium, titanium, zirconium, and one or more precious metals, and wherein an acetic acid productivity is higher than about 50 g/kg cat.h after 100 hours of time on stream.

A forty seventh aspect can include the method of the forty sixth aspect, wherein an acetic acid selectivity is higher than about 30 mole percent based on the total moles of a feed stream.

A forty eighth aspect can include the method of the forty sixth or forty seventh aspect, further comprising: removing a gas phase stream from the reactor; separating the gas phase stream to produce a lights recycle stream and a gas stream; and recycling at least a portion of one of: the gas phase stream, the lights recycle stream, or the gas stream to the reactor.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. For example, features described as method steps may have corresponding elements in the system embodiments described above, and vice versa. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically, and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as comprises, includes, and having should be understood to provide support for narrower terms such as consisting of, consisting essentially of, and comprised substantially of. Use of the term “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1. A method for converting one or more hydrocarbons to oxygenates,
the method comprising:
feeding a fluid comprising one or more light alkanes, an oxidative gas comprising oxygen and nitrogen, and water to a reactor system, wherein the reactor system comprises a reactor containing at least one catalyst, wherein the at least one catalyst comprises one or more oxides of molybdenum, vanadium, niobium, cerium, titanium, zirconium, silicon or a combination thereof, and one or more precious metals comprising ruthenium, rhodium, palladium, osmium, iridium, platinum, gold, or any combination thereof,
producing one or more oxygenates from the one or more light alkanes in the reactor system as an output stream;
separating the one or more oxygenates from the output stream in a separation system in fluid communication with the reactor system, and
recycling at least a portion of any unconverted light alkanes from the separation system to the reactor system.

2. The method of claim 1, where the one or more oxygenates comprise acetic acid.

3. (canceled)

4. The method of claim 1, further comprising:
separating a gas phase stream and a liquid phase stream from the output stream in the separation system, wherein recycling the unconverted light alkanes comprises recycling at least a portion of the gas phase stream to the reactor system;
passing the liquid phase stream to a liquid separation system.

5-6. (canceled)

7. The method of claim 1, wherein the one or more light alkanes comprises methane, ethane, propane, and butane.

8. The method of claim 1, further comprising: selectively converting the one or more light alkanes comprising ethane to the one or more oxygenates in the presence of a gas comprising oxygen and water.

9. The method of claim 1, further comprising: operating the reactor system at a temperature of about 150° C. to about 600° C.

10. The method of claim 1, further comprising: operating the reactor system at a pressure of about 3 bar to about 100 bar.

11. The method of claim 1, further comprising: operating a gas hourly space velocity of the reactor system of about 50 h⁻¹ to about 20,000 h⁻¹.

12. The method of claim 1, wherein the at least one catalyst comprises a material having a formula
DᵤAₓB_{y}C;
wherein A is vanadium oxide and x is in a range of from 5 to 25 mass percentage;
wherein B is a mixture of molybdenum oxide and niobium oxide and y is in a range of from 25 to 75 mass percentage;
wherein C is at least one of titania, ceria, silica, or zirconia and z is in a range from 25 to 75 mass percentage;
wherein D is at least one of ruthenium, rhodium, palladium osmium, iridium, platinum, or gold and u is in a range of from 0.01 to 1 mass percentage.

13. (canceled)

14. The method of claim 1, wherein the reactor system comprises at least two reactors in series with a cooler after a first reactor of the at least two reactors to cool an effluent of the first reactor before passing the effluent to a second reactor.

15. The method of claim 1, wherein the reactor system comprises at least two isothermal-reactors in series with a condenser and phase separator after a first reactor of the at least two reactors to condense and phase separate at least a portion of an effluent of the first reactor before passing the effluent to a second reactor.

16. The method of claim 4, further comprising:
producing a water stream from the liquid phase stream in the liquid separation system; and
recycling at least a portion of the water stream to the reactor system, wherein the water stream comprises a portion of the one or more oxygenates.

17. The method of claim 16, wherein the one or more oxygenates comprise acetic acid, and wherein producing the water stream comprises: distilling the water stream in a distillation column without the use of an entrainer.

18. The method of claim 16, wherein the one or more oxygenates comprise acetic acid, and wherein the method further comprises:
distilling the liquid phase using azeotropic distillation to remove at least a portion of the liquid phase from the acetic acid and produce an acetic acid product and a liquid stream;
providing an entrainer within a distillation column during distilling;
providing the liquid stream after distilling to a decanter for recycling an organic phase to the distillation column and an aqueous phase to another distillation column for separating a water stream; and
separating the acetic acid product during the distillation.

19. The method of claim 18, wherein the entrainer comprises ethyl acetate, propyl acetate, butyl acetate, or any combination thereof.

20.-23. (canceled)

24. A system for converting one or more hydrocarbons, the system comprising:
a reactor system, wherein the reactor system comprises at least one reactor containing at least one catalyst, the at least one catalyst comprises one or more oxides of molybdenum, vanadium, niobium, cerium, titanium, zirconium, silicon, or a combination thereof and one or more precious metals comprising ruthenium, rhodium, palladium, osmium, iridium, platinum, gold, or any combination thereof, and wherein the reactor system is configured to react one or more light alkanes in the presence of the catalyst to produce one or more oxygenates comprising acetic acid in an output stream;
a separation system in fluid communication with the reactor system, where the separation system is configured to separate the one or more oxygenates from the output stream and recycle at least a portion of any unconverted light alkanes to the reactor system.

25-28. (canceled)

29. The system of claim 24, wherein the reactor system comprises at least two reactors in series and a cooler fluidly coupled between a first reactor of the at least two reactors and a second reactor of the at least two reactors.

30-31. (canceled)

32. The system of claim 24, further comprising:
a gas separation system and a liquid separation system disposed downstream of the reactor system, wherein the liquid separation system is configured to produce a water stream from a liquid phase; and
a recycle line configured to recycle at least a portion of the water stream to the reactor system.

33. The system of claim 32, wherein the one or more oxygenates comprise acetic acid, and wherein the system further comprises:
a distillation column, wherein the distillation system comprises an entrainer within the distillation column, wherein the distillation column is configured to distill the liquid phase using azeotropic distillation to remove at least a portion of the liquid phase from the acetic acid and produce an acetic acid product and a liquid stream;
a decanter configured to receive the liquid stream, wherein the decanter is configured to provide an organic phase and recycle the organic phase to the distillation column and to provide an aqueous phase to a second distillation column; and
the second distillation column, wherein the second distillation column is configured to generate the acetic acid product.

34. The system of claim 32, wherein the gas separation system comprises:
at least one of a gas recycle line with a purge vent, a membrane assembly, a carbon dioxide scrubber, or an absorber configured to receive a gas phase stream from the gas separation system.

35. The method of claim 4, wherein the gas phase stream comprises air, nitrogen, oxygen, ethylene, ethane, methane, propane, butane(s), carbon monoxide, carbon dioxide, water, or the combinations thereof.

36. The method of claim 4, wherein the separation system comprises of at least one of gas recycle to the reactor after a purge, a membrane assembly, a carbon dioxide scrubber, or an absorber.

37. The method of claim 1, wherein the reactors in the reactor system is operated in an adiabatic mode or an isothermal mode.

38. The method of claim 1, wherein recycling the unconverted light alkanes, nitrogen, and carbon dioxide from the separation system to the reactor system comprises:
diluting the fluid fed into the reactor system; and
controlling a temperature of the reactor using the recycled unconverted light alkanes.

39. The system of claim 24, wherein the at least one reactor is configured to operate in an adiabatic mode or an isothermal mode.
